(19) United States
(12) Patent Application Publication
(10) Pub. No.: US 2024/0180894 A1
(43) Pub. Date: Jun. 6, 2024

(54) METHODS OF TREATING MENTAL AND/OR MOOD DISORDERS USING 2-BROMO-LSD ALONE OR IN COMBINATION WITH A MTOR INHIBITOR

(71) Applicant: BeTTERLIFE PHARMA INC., Vancouver (CA)

(72) Inventor: Justin KIRKLAND, Champaign, IL (US)

(21) Appl. No.: 18/283,373

(22) PCT Filed: Mar. 3, 2022
(86) PCT No.: PCT/CA2022/050427

§ 371 Date: Sep. 21, 2023

Related U.S. Application Data
(60) Provisional application No. 63/163,954, filed on Mar. 22, 2021.

(51) Int. Cl. A61K 31/48; A61K 9/00; A61K 9/08; A61K 9/20; A61K 9/48; A61K 31/436

(52) U.S. Cl. CPC A61K 31/48; A61K 9/0053; A61K 9/08; A61K 9/2009; A61K 9/2013; A61K 9/2018; A61K 9/2027; A61K 9/2054; A61K 9/485; A61K 9/4866; A61K 31/436

(57) ABSTRACT

Methods for treatment of mental and/or mood disorders in a subject are disclosed. The methods comprise administration to a subject in need thereof an effective amount of 2-bromo-LSD and/or a mTOR inhibitor. Also disclosed are pharmaceutical compositions that comprise 2-bromo-LSD and/or a mTOR inhibitor as well as dosage formulations and dosage regimes thereof.

### FIELD

Methods and compositions for treatment of mental and/or mood disorders comprising use of 2-bromo-LSD and/or a mTOR inhibitor.

### BACKGROUND

Major depressive disorder (MDD) is a psychiatric disorder which has a lifetime prevalence of around 8%. MDD is characterized by increased medical morbidity, mortality, functional impairment, reduced quality of life, substantial health-care costs, and an increased risk of suicide, loss of interest or pleasure, disturbed sleep or appetite, low energy, and feelings of guilt or low self-worth (Greden et al, J Clin Psychiat. 2001, 62:26-31; Tranter et al, J Psychiat Neurosci. 2002, 27:241-247; Uher et al, Depress Anxiety. 2014, 31:459-471). MDD is one of the most common mental disorders worldwide, with a life time prevalence of 16.2% and a 12-month prevalence of 6.6% in developed countries (Trivedi et al, CNS Spectr. 2007, 12:1-27). According to the World Health Organization (WHO, 2010), MDD carries the heaviest burden of disability among mental and behavioral disorders. The findings from the 2014 National Survey on Drug Use and Health (NSDUH) revealed that in 2014, 11.4 percent of youths aged 12 to 17 (2.8 million adolescents) had a major depressive episode (MDE) in the past year (Center for Behavioral Health Statistics and Quality, 2015). Youths aged 12 to 17 in 2014 who had a past year MDE were more likely than those without a past year MDE to have used any illicit drugs in the past year (33.0 vs. 15.2 percent). Initial antidepressant therapy reduces symptoms of depression in patients, but only 50-60% of patients with major depressive disorder respond to therapy (Nierenberg and DeCecco, J Clin Psychiat. 2001, 62:5-9). Furthermore, between 30 and 40% of patients who suffer from MDD never achieve symptom resolution with standard antidepressant treatment (Amsterdam and HornigRohan, Psychiatr Clin North Am. 1996, 19:371-386; Nierenberg and Amsterdam, J Clin Psychiat. 1990, 51:39-47).

### SUMMARY OF THE INVENTION

Needed are treatments for subjects having depression, in aspects major depressive disorder, in aspects symptoms of major depressive disorder, in aspects side effects of major depressive disorder- and any of the foregoing alone or in combination. A first aspect of the present disclosure is a method of treating major depressive disorder in a subject in need thereof, wherein the subject has been diagnosed with the major depressive disorder, comprising: providing an mTOR inhibitor; providing 2-Bromo-LSD; administering a therapeutically effective amount of the mTOR inhibitor to the subject; and administering a therapeutically effective amount of the 2-Bromo-LSD to the subject, so as to treat the major depressive disorder. In some embodiments, the mTOR inhibitor is a small molecule. In some embodiments, the mTOR inhibitor is a macrolide. In some embodiments, the mTOR inhibitor is an antibody. In some embodiments, the mTOR inhibitor is a nucleic acid molecule capable of down-regulating mTOR activity.

In some embodiments, the mTOR inhibitor is selected from the group consisting of rapamycin (sirolimus), rapamycin derivatives, everolimus, ABT-578, tacrolimus (FK 506), ABT-578, AP-23675, BEZ-235, OSI-027, QLT-0447, ABI-009, BC-210, salirasib, TAFA-93, deforolimus (AP-23573), temsirolimus, 2-(4-Amino-1-isopropyl-1H-pyrazolo[3,4-d]pyrimidin-3-yl)-1H-indol-5-ol (PP242), and AP-23841. In some embodiments, the mTOR inhibitor is rapamycin. In some embodiments, the mTOR inhibitor is a product of rapamycin. In some embodiments, the mTOR inhibitor is a rapamycin derivative having at least one of a C-40, C-16 or C-32 position modification as compared with rapamycin.

In some embodiments, the mTOR inhibitor and the 2-Bromo-LSD are administered concurrently. In some embodiments, the mTOR inhibitor and the 2-Bromo-LSD are administered sequentially.

In some embodiments, the mTOR inhibitor and the 2-Bromo-LSD are provided in the same unit dosage form.

In some embodiments, the mTOR inhibitor and the 2-Bromo-LSD are administered within 12 hours of each other. In some embodiments, the mTOR inhibitor and the 2-Bromo-LSD are administered within 6 hours of each other. In some embodiments, the mTOR inhibitor and the 2-Bromo-LSD are administered within 4 hours of each other. In some embodiments, the mTOR inhibitor and the 2-Bromo-LSD are administered within 2 hours of each other.

In some embodiments, the therapeutically effective amount of the mTOR inhibitor ranges from between about 0.1 to about 100 mg/day. In some embodiments, the therapeutically effective amount of the mTOR inhibitor ranges from between about 0.1 to about 50 mg/day. In some embodiments, the therapeutically effective amount of the mTOR inhibitor ranges from between about 0.1 to about 25 mg/day. In some embodiments, the therapeutically effective amount of the mTOR inhibitor ranges from between about 0.5 to about 10 mg/day. In some embodiments, the therapeutically effective amount of the mTOR inhibitor is 0.1-0.5 mg/day. 0.5-1.0 mg/day, 1.0-1.5 mg/day, 1.5-2 mg/day, 2.0-2.5 mg/day, 2.5-5 mg/day, 5-10 mg/day, 10-15 mg/day, 15-20 mg/day, 20-25 mg/day, 25-30 mg/day, 30-35 mg/day, 35-40 mg/day, 40-45 mg/day, 45-50 mg/day, 50-55 mg/day, 55-60 mg/day, 60-65 mg/day, 65-70 mg/day, 70-75 mg/day, 75-80 mg/day, 80-85 mg/day, 85-90 mg/day, 90-95 mg/day or 95-100 mg/day.

In some embodiments, the mTOR inhibitor is administered once weekly. In some embodiments, the mTOR inhibitor is administered twice weekly. In some embodiments, the mTOR inhibitor is administered three times per week. In some embodiments, the mTOR inhibitor is administered four times per week.

In some embodiments, the mTOR inhibitor and/or 2-Bromo-LSD are administered intravenously, intramuscularly, intraperitoneally, orally or via inhalation.

A second aspect of the present disclosure is a method of treating major depressive disorder in a subject in need thereof, wherein the subject has been diagnosed with the major depressive disorder, comprising: providing an mTOR inhibitor, wherein the mTOR inhibitor is selected from the group consisting of (i) temsirolimus (CCI-779) or a pharmaceutical equivalent, analog, derivative or a salt thereof, (ii) Everolimus (RAD001) or a pharmaceutical equivalent, analog, derivative or a salt thereof, and (iii) Rapamycin or a pharmaceutical equivalent, analog, derivative or a salt thereof; providing 2-Bromo-LSD; administering a therapeutically effective amount of the mTOR inhibitor to the subject; and administering a therapeutically effective amount of the 2-Bromo-LSD to the subject, so as to treat the major depressive disorder. In some embodiments, the mTOR inhibitor and the 2-Bromo-LSD are administered concurrently. In some embodiments, the mTOR inhibitor and the 2-Bromo-LSD are administered sequentially.

In some embodiments, the mTOR inhibitor and the 2-Bromo-LSD are administered within 12 hours of each other. In some embodiments, the mTOR inhibitor and the 2-Bromo-LSD are administered within 6 hours of each other. In some embodiments, the mTOR inhibitor and the 2-Bromo-LSD are administered within 4 hours of each other. In some embodiments, the mTOR inhibitor and the 2-Bromo-LSD are administered within 2 hours of each other.

In some embodiments, the effective amount of the mTOR inhibitor is 0.1-0.5 mg/day. 0.5-1.0 mg/day, 1.0-1.5 mg/day, 1.5-2 mg/day, 2.0-2.5 mg/day, 2.5-5 mg/day, 5-10 mg/day, 10-15 mg/day, 15-20 mg/day, 20-25 mg/day, 25-30 mg/day, 30-35 mg/day, 35-40 mg/day, 40-45 mg/day, 45-50 mg/day, 50-55 mg/day, 55-60 mg/day, 60-65 mg/day, 65-70 mg/day, 70-75 mg/day, 75-80 mg/day, 80-85 mg/day, 85-90 mg/day, 90-95 mg/day or 95-100 mg/day.

In some embodiments, the mTOR inhibitor and/or 2-Bromo-LSD are administered intravenously, intramuscularly, intraperitoneally, orally or via inhalation.

A third aspect of the present disclosure is a method of lessening or alleviating the severity of a major depressive disorder in a subject in need thereof, wherein the subject has been diagnosed with the major depressive disorder, comprising: providing an mTOR inhibitor; providing 2-Bromo-LSD; administering a therapeutically effective amount of the mTOR inhibitor to the subject; and administering a therapeutically effective amount of the 2-Bromo-LSD to the subject, so as to lessen or alleviate the severity of the major depressive disorder. In some embodiments, the mTOR inhibitor is a small molecule. In some embodiments, the mTOR inhibitor is a macrolide. In some embodiments, the mTOR inhibitor is an antibody. In some embodiments, the mTOR inhibitor is a nucleic acid molecule capable of down-regulating mTOR activity.

In some embodiments, the mTOR inhibitor is selected from the group consisting of rapamycin (sirolimus), rapamycin derivatives, everolimus, ABT-578, tacrolimus (FK 506), ABT-578, AP-23675, BEZ-235, OSI-027, QLT-0447, ABI-009, BC-210, salirasib, TAFA-93, deforolimus (AP-23573), temsirolimus, 2-(4-Amino-1-isopropyl-1H-pyrazolo[3,4-d]pyrimidin-3-yl)-1H-indol-5-ol (PP242), and AP-23841. In some embodiments, the mTOR inhibitor is rapamycin. In some embodiments, the mTOR inhibitor is a product of rapamycin. In some embodiments, the mTOR inhibitor is a rapamycin derivative having at least one of a C-40, C-16 or C-32 position modification as compared with rapamycin.

In some embodiments, the mTOR inhibitor and the 2-Bromo-LSD are administered concurrently. In some embodiments, the mTOR inhibitor and the 2-Bromo-LSD are administered sequentially.

In some embodiments, the mTOR inhibitor and the 2-Bromo-LSD are provided in the same unit dosage form.

A fourth aspect of the present disclosure is a method of reducing at least one symptom of a major depressive disorder in a subject in need thereof, wherein the subject has been diagnosed with the major depressive disorder, comprising: providing an mTOR inhibitor; providing 2-Bromo-LSD; administering a therapeutically effective amount of the mTOR inhibitor to the subject; and administering a therapeutically effective amount of the 2-Bromo-LSD to the subject, so as to reduce the at least one symptom of the major depressive disorder. In some embodiments, the mTOR inhibitor is a small molecule. In some embodiments, the mTOR inhibitor is a macrolide. In some embodiments, the mTOR inhibitor is an antibody. In some embodiments, the mTOR inhibitor is a nucleic acid molecule capable of down-regulating mTOR activity.

In some embodiments, the mTOR inhibitor is selected from the group consisting of rapamycin (sirolimus), rapamycin derivatives, everolimus, ABT-578, tacrolimus (FK 506), ABT-578, AP-23675, BEZ-235, OSI-027, QLT-0447, ABI-009, BC-210, salirasib, TAFA-93, deforolimus (AP-23573), temsirolimus, 2-(4-Amino-1-isopropyl-1H-pyrazolo[3,4-d]pyrimidin-3-yl)-1H-indol-5-ol (PP242), and AP-23841. In some embodiments, the mTOR inhibitor is rapamycin. In some embodiments, the mTOR inhibitor is a product of rapamycin. In some embodiments, the mTOR inhibitor is a rapamycin derivative having at least one of a C-40, C-16 or C-32 position modification as compared with rapamycin.

In some embodiments, the mTOR inhibitor and the 2-Bromo-LSD are administered concurrently. In some embodiments, the mTOR inhibitor and the 2-Bromo-LSD are administered sequentially.

In some embodiments, the mTOR inhibitor and the 2-Bromo-LSD are provided in the same unit dosage form.

A fifth aspect of the present disclosure is a method of lessening or alleviating the severity of a major depressive disorder in a subject in need thereof, wherein the subject has been diagnosed with the major depressive disorder, comprising: providing an mTOR inhibitor, wherein the mTOR inhibitor is selected from the group consisting of (i) temsirolimus (CCI-779) or a pharmaceutical equivalent, analog, derivative or a salt thereof, (ii) Everolimus (RAD001) or a pharmaceutical equivalent, analog, derivative or a salt thereof, and (iii) Rapamycin or a pharmaceutical equivalent, analog, derivative or a salt thereof; providing 2-Bromo-LSD; administering a therapeutically effective amount of the mTOR inhibitor to the subject; and administering a therapeutically effective amount of the 2-Bromo-LSD to the subject. In some embodiments, the mTOR inhibitor and the 2-Bromo-LSD are administered concurrently. In some embodiments, the mTOR inhibitor and the 2-Bromo-LSD are administered sequentially. In some embodiments, the mTOR inhibitor and the 2-Bromo-LSD are administered within 12 hours of each other. In some embodiments, the mTOR inhibitor and the 2-Bromo-LSD are administered within 6 hours of each other. In some embodiments, the mTOR inhibitor and the 2-Bromo-LSD are administered within 4 hours of each other. In some embodiments, the mTOR inhibitor and the 2-Bromo-LSD are administered within 2 hours of each other.

In some embodiments, the effective amount of the mTOR inhibitor is 0.1-0.5 mg/day. 0.5-1.0 mg/day, 1.0-1.5 mg/day, 1.5-2 mg/day, 2.0-2.5 mg/day, 2.5-5 mg/day, 5-10 mg/day, 10-15 mg/day, 15-20 mg/day, 20-25 mg/day, 25-30 mg/day, 30-35 mg/day, 35-40 mg/day, 40-45 mg/day, 45-50 mg/day, 50-55 mg/day, 55-60 mg/day, 60-65 mg/day, 65-70 mg/day, 70-75 mg/day, 75-80 mg/day, 80-85 mg/day, 85-90 mg/day, 90-95 mg/day or 95-100 mg/day. In some embodiments, the mTOR inhibitor and/or 2-Bromo-LSD are administered intravenously, intramuscularly, intraperitoneally, orally or via inhalation.

A sixth aspect of the present disclosure is a method of treating a mental disorder and/or a mood disorder in a subject in need thereof, wherein the subject has been diagnosed with the major depressive disorder, comprising: providing an mTOR inhibitor; providing 2-Bromo-LSD; administering a therapeutically effective amount of the mTOR inhibitor to the subject; and administering a therapeutically effective amount of the 2-Bromo-LSD to the subject, so as to treat the mental disorder and/or the mood disorder. In some embodiments the mental disorder and/or the mood disorder is selected from the group consisting of Attention Deficit Hyperactivity Disorder, Obsessive-Compulsive Disorder, Atypical Depression, Melancholid Depression, Psychotic major depression, Catatonic Depression, Postpartum Depression, Premenstrual dysphoric disorder, Seasonal affective disorder, Dysthymia Double Depression, Depressive Disorder Not Otherwise Specified, Depressive personality disorder, Recurrent brief depression, and Minor Depressive disorder.

In some embodiments, the mTOR inhibitor is a small molecule. In some embodiments, the mTOR inhibitor is a macrolide. In some embodiments, the mTOR inhibitor is an antibody. In some embodiments, the mTOR inhibitor is a nucleic acid molecule capable of down-regulating mTOR activity.

In some embodiments, the mTOR inhibitor is selected from the group consisting of rapamycin (sirolimus), rapamycin derivatives, everolimus, ABT-578, tacrolimus (FK 506), ABT-578, AP-23675, BEZ-235, OSI-027, QLT-0447, ABI-009, BC-210, salirasib, TAFA-93, deforolimus (AP-23573), temsirolimus, 2-(4-Amino-1-isopropyl-1H-pyrazolo[3,4-d]pyrimidin-3-yl)-1H-indol-5-ol (PP242), and AP-23841. In some embodiments, the mTOR inhibitor is rapamycin. In some embodiments, the mTOR inhibitor is a product of rapamycin. In some embodiments, the mTOR inhibitor is a rapamycin derivative having at least one of a C-40, C-16 or C-32 position modification as compared with rapamycin.

In some embodiments, the mTOR inhibitor and the 2-Bromo-LSD are administered concurrently. In some embodiments, the mTOR inhibitor and the 2-Bromo-LSD are administered sequentially.

In some embodiments, the mTOR inhibitor and the 2-Bromo-LSD are provided in the same unit dosage form.

In some embodiments, the mTOR inhibitor and the 2-Bromo-LSD are administered within 12 hours of each other. In some embodiments, the mTOR inhibitor and the 2-Bromo-LSD are administered within 6 hours of each other. In some embodiments, the mTOR inhibitor and the 2-Bromo-LSD are administered within 4 hours of each other. In some embodiments, the mTOR inhibitor and the 2-Bromo-LSD are administered within 2 hours of each other.

In some embodiments, the therapeutically effective amount of the mTOR inhibitor ranges from between about 0.1 to about 100 mg/day. In some embodiments, the therapeutically effective amount of the mTOR inhibitor ranges from between about 0.1 to about 50 mg/day. In some embodiments, the therapeutically effective amount of the mTOR inhibitor ranges from between about 0.1 to about 25 mg/day. In some embodiments, the therapeutically effective amount of the mTOR inhibitor ranges from between about 0.5 to about 10 mg/day. In some embodiments, the therapeutically effective amount of the mTOR inhibitor is 0.1-0.5 mg/day. 0.5-1.0 mg/day, 1.0-1.5 mg/day, 1.5-2 mg/day, 2.0-2.5 mg/day, 2.5-5 mg/day, 5-10 mg/day, 10-15 mg/day, 15-20 mg/day, 20-25 mg/day, 25-30 mg/day, 30-35 mg/day, 35-40 mg/day, 40-45 mg/day, 45-50 mg/day, 50-55 mg/day, 55-60 mg/day, 60-65 mg/day, 65-70 mg/day, 70-75 mg/day, 75-80 mg/day, 80-85 mg/day, 85-90 mg/day, 90-95 mg/day or 95-100 mg/day.

In some embodiments, the mTOR inhibitor is administered once weekly. In some embodiments, the mTOR inhibitor is administered twice weekly. In some embodiments, the mTOR inhibitor is administered three times per week. In some embodiments, the mTOR inhibitor is administered four times per week.

In some embodiments, the mTOR inhibitor and/or 2-Bromo-LSD are administered intravenously, intramuscularly, intraperitoneally, orally or via inhalation.

A seventh aspect of the present disclosure is a composition comprising: (i) a therapeutically effective dose of an mTOR inhibitor; (ii) a therapeutically effective dose of 2-Bromo-LSD; and (iii) a pharmaceutically acceptable carrier or excipient.

An eighth aspect of the present disclosure is a kit comprising: (i) a therapeutically effective dose of an mTOR inhibitor; and (ii) a therapeutically effective dose of 2-Bromo-LSD. In some embodiments. In some embodiments, the therapeutically effective dose of the mTOR inhibitor is provided in a first container and the therapeutically effective dose of the 2-Bromo-LST are provided in a second container, the daily (or other periodic) dosages may be arranged for proper sequential or simultaneous administration. The kit or container containing a plurality of dosage units, adapted for successive daily administration, each dose comprising at least one of the therapeutic agents of the present invention. In one embodiment, the kit contains a plurality of dosage/doses to be administered daily or weekly where at least one of the dosages/doses is administered via a different route than the other. In some embodiments, the kit further comprises instructions for the administration of the therapeutically effective dose of an mTOR inhibitor and the therapeutically effective dose of 2-Bromo-LSD. In some embodiments, the therapeutically effective dose of the mTOR inhibitor and the therapeutically effective dose of the 2-Bromo-LSD are both provided for oral administration, e.g. orally dissolving tablets, capsules, tablets, etc. In some embodiments, the mTOR inhibitor is provided as a transdermal patch for continuous administration; and the 2-Bromo-Lst is provided in an oral dosage form. The present methods, kits, combinations, and compositions can also be used in “combination therapy” with another pharmaceutical agent.

A ninth aspect of the present disclosure is a method of prolonging an antidepressant effect of 2-Bromo-LSD, comprising: providing an mTOR inhibitor; providing 2-Bromo-LSD; administering a therapeutically effective amount of the mTOR inhibitor to a subject; and administering a therapeutically effective amount of the 2-Bromo-LSD to the subject, so as to extend the antidepressant effect of the 2-Bromo-LSD. In some embodiments, the mTOR inhibitor is a small molecule. In some embodiments, the mTOR inhibitor is a macrolide. In some embodiments, the mTOR inhibitor is an antibody. In some embodiments, the mTOR inhibitor is a nucleic acid molecule capable of down-regulating mTOR activity.

In some embodiments, the mTOR inhibitor is selected from the group consisting of rapamycin (sirolimus), rapamycin derivatives, everolimus, ABT-578, tacrolimus (FK 506), ABT-578, AP-23675, BEZ-235, OSI-027, QLT-0447, ABI-009, BC-210, salirasib, TAFA-93, deforolimus (AP-23573), temsirolimus, 2-(4-Amino-1-isopropyl-1H-pyrazolo[3,4-d]pyrimidin-3-yl)-1H-indol-5-ol (PP242), and AP-23841. In some embodiments, the mTOR inhibitor is rapamycin. In some embodiments, the mTOR inhibitor is a product of rapamycin. In some embodiments, the mTOR inhibitor is a rapamycin derivative having at least one of a C-40, C-16 or C-32 position modification as compared with rapamycin.

In some embodiments, the mTOR inhibitor and the 2-Bromo-LSD are administered concurrently. In some embodiments, the mTOR inhibitor and the 2-Bromo-LSD are administered sequentially. In some embodiments, the mTOR inhibitor is administered first and the 2-Bromo-LSD is administered second. In some embodiments, the mTOR inhibitor is administered 1 hour prior to the administration of the 2-Bromo-LSD. In some embodiments, the mTOR inhibitor is administered 2 hours prior to the administration of the 2-Bromo-LSD. In some embodiments, the mTOR inhibitor is administered 3 hours prior to the administration of the 2-Bromo-LSD. In some embodiments, the mTOR inhibitor is administered 4 hours prior to the administration of the 2-Bromo-LSD. In some embodiments, the mTOR inhibitor is administered 6 hours prior to the administration of the 2-Bromo-LSD. In some embodiments, the mTOR inhibitor is administered 12 hours prior to the administration of the 2-Bromo-LSD.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either” “one of,” “only one of” or “exactly one of” “Consisting essentially of,” when used in the claims, shall have its ordinary meaning as used in the field of patent law.

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the terms “co-administration” or “co-administering” are meant to refer to a combination therapy in which two or more agents are administered to a patient or subject by any administration route. Co-administration of agents may also be referred to as combination therapy or combination treatment. The agents may be in the same dosage formulation or separate formulations. For combination treatment with more than one active agent, where the active agents are in separate dosage formulations, the active agents can be administered concurrently, or they each can be administered at separately staggered times. The agents of a combination treatment may be administered simultaneously or sequentially (e.g., one agent may directly follow administration of the other or the agents may be given episodically, e.g., one can be given at one time followed by the other at a later time, e.g., within a week), as long as they are given in a manner sufficient to allow both agents to achieve effective concentrations in the body. The agents may also be administered by different routes, e.g., one agent may be administered intravenously while a second agent is administered intramuscularly, intravenously, or orally.

As used herein, the phrases “effective amount” or “therapeutically effective amount” (used interchangeably herein) refer to the amount of a composition or formulation described herein that will elicit the diagnostic, biological or medical response of a tissue, system, animal, or human that is being sought by the researcher, veterinarian, medical doctor, or other clinician.

As used herein, the term “mTOR inhibitor” refers to a compound or a ligand that inhibits at least one activity of an mTOR, such as the serine/threonine protein kinase activity on at least one of its substrates (e.g., p70S6 kinase 1, 4E-BPT, AKT/PKB and eEF2). mTOR inhibitors include, but are not limited to small molecule, antibody, peptide, and nucleic acid inhibitors. For example, an mTOR inhibitor can be a molecule that inhibits the kinase activity of mTOR or inhibits binding of mTOR to a ligand. Inhibitors of mTOR also include molecules that down-regulate expression of mTOR, such as an antisense compound. Examples of mTOR inhibitors include, without limitation, rapamycin (sirolimus), rapamycin derivatives, CI-779, everolimus (Certican™), ABT-578, tacrolimus (FK 506), ABT-578, AP-23675, BEZ-235, OSI-027, QLT-0447, ABI-009, BC-210, salirasib, TAFA-93, deforolimus (AP-23573), temsirolimus (Torisel™), 2-(4-Amino-1-isopropyl-1H-pyrazolo[3,4-d]pyrimidin-3-yl)-1H-indol-5-ol (PP242) and AP-23841. In some embodiments, the mTOR inhibitor is rapamycin or a rapamycin analog. Yet other mTOR inhibitors are described herein.

As used herein, the terms “pharmaceutically acceptable excipient,” “carrier,” or “diluent” refer to pharmaceutical components which do not alter the therapeutic properties of an active agent with which it is administered. One exemplary pharmaceutically acceptable carrier substance is physiological saline. For instance, the pharmaceutically acceptable carrier can include sodium chloride (e.g., 150 mM sodium chloride) and sodium phosphate (e.g., 25 mM sodium phosphate). Other physiologically acceptable excipients, carriers, and diluents, and their formulations, are known to those skilled in the art and described, e.g., in Remington: The Science and Practice of Pharmacy (22nd Ed), Allen (2012). For instance, a pharmaceutically acceptable excipient, carrier, or diluent can include dibasic sodium phosphate, heptahydrate; monobasic sodium phosphate, monohydrate; and sodium chloride at a pH between 7.2 and 7.6.

As used herein, the term “pharmaceutical composition” it is meant a composition containing an active agent as described herein, formulated with at least one pharmaceutically acceptable excipient, carrier, or diluent. The pharmaceutical composition can be manufactured or sold with the approval of a governmental regulatory agency as part of a therapeutic regimen for the treatment or prevention of a disease or event in a patient (e.g., an infant with HPP, such as an infant having perinatal-onset HPP, or an infant having infantile-onset HPP, or juvenile-onset HPP, or a patient having childhood-onset HPP). Pharmaceutical compositions can be formulated, for example, for subcutaneous administration, intravenous administration (e.g., as a sterile solution free of particulate emboli and in a solvent system suitable for intravenous use), for oral administration (e.g., a tablet, capsule, caplet, gelcap, or syrup), for transdermal administration, or any other formulation described herein, e.g., in unit dosage form.

As used herein, the term “rapamycin” refers to a small molecule with known immunosuppressive and anti-proliferative properties. Rapamycin, also known as sirolimus, is a macrolide that was first discovered as a product of the bacterium Streptomyces hygroscopicus. Rapamycin binds and inhibits the activity of mTOR. The chemical formula of rapamycin is C51H79NO13 and the International Union of Pure and Applied Chemistry (IUPAC) name is (3S,6R,7E,9R,10R,12R,14S,15E,17E,19E,21S,23S,26R,27R,34aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]-oxaazacyclohentriacontine-1,5,11,28,29(4H,6H,31H)-pentone.

As used herein, the term “subject” refers to any animal subject including laboratory animals (e.g., primates, rats, mice), livestock (e.g., cows, sheep, goats, pigs, turkeys, chickens), household pets (e.g., dogs, cats, rodents, etc.), and humans. The subject can be a mammal. Typically, the mammal is a human (Homo sapiens).

As used herein, the terms “treatment,” “treating,” or “treat,” with respect to a specific condition (e.g. major depressive disorder), refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. The effect can be a decrease/lessening/reduction of one or more symptoms of MDD. In some embodiments, symptoms associated with depression/MDD include, but are not limited to, persistent anxious or sad feelings, feelings of helplessness, hopelessness, pessimism, and/or worthlessness, low energy, restlessness, irritability/short temper, fatigue, loss of interest in pleasurable activities or hobbies, excessive sleeping, overeating, appetite loss, insomnia, memory loss, crying uncontrollably, thoughts of suicide, or suicide attempts. In addition, patients suffering from any form of depression often experience anxiety. It is expected that the methods of the present condition can be used to treat anxiety or any of the symptoms thereof. In some embodiments, presence, severity, frequency, and duration of symptoms of depression vary on a case to case basis. In some embodiments, a complication of MDD may be alleviated such as pain or physical illness, alcohol and drug misuse, societal isolation, self-mutilation and panic/anxiety disorder. “Treatment”, as used herein, covers any treatment of a disease or disorder in a subject, particularly in a human, and includes: (a) preventing the disease or disorder from occurring in a subject which may be predisposed to the disease but has not yet been diagnosed as having it; (b) inhibiting the disease or disorder, i.e., arresting its development; and (c) relieving or alleviating the disease or disorder, i.e., causing regression of the disease or disorder and/or relieving one or more disease or disorder symptoms. “Treatment” can also encompass delivery of an agent or administration of a therapy in order to provide for a pharmacologic effect, even in the absence of a disease, disorder, or condition. The term “treatment” is used in some embodiments to refer to administration of a compound of the present disclosure to mitigate a disease or a disorder in a host, preferably in a mammalian subject, more preferably in humans. Thus, the term “treatment” can include preventing a disorder from occurring in a host, particularly when the host is predisposed to acquiring the disease but has not yet been diagnosed with the disease; inhibiting the disorder; and/or alleviating or reversing the disorder. As far as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure can occur prior to onset of a disease. The term does not mean that the disease state must be completely avoided.

### Overview

It would be desirable to treat subjects suffering from mental and/or mood disorders with an improved pharmaceutical composition that is able to resolve symptoms even in subjects who have failed prior therapy. As such, it is an object of the present disclosure to provide a method for treating a mental disorder and/or a mood disorder in a subject in need of treatment thereof. In some embodiments, the present disclosure relates to a method of treating or therapeutically managing a mental disorder or a mood disorder in a subject in need of treatment thereof, the method comprising administering an effective amount of 2-bromolysergic acid diethylamide (hereinafter “2-Bromo-LSD”) or a derivative or salt thereof, or a pharmaceutical composition comprising 2-Bromo-LSD or a derivative or salt thereof 2-Bromo-LSD and its method of synthesis are described in U.S. Pat. Nos. 9,868,732 and 10,377,752, the disclosures of which are hereby incorporated by reference herein in their entireties. The chemical structure of 2-Bromo-LSD is depicted below.

Another object of the present disclosure includes co-administering one or more mTOR inhibitors in conjunction with the administration of 2-Bromo-LSD. In some embodiments, the co-administration of the one or more mTOR inhibitors and 2-Bromo-LSD comprises concurrent administration. In some embodiments, the co-administration of the one or more mTOR inhibitors and 2-Bromo-LSD comprises sequential administration.

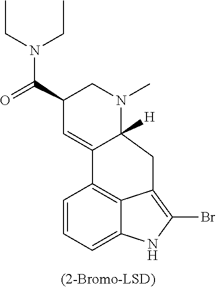 [SMILES: CCN(CC)C(=O)[C@@H]1C=C2c3cccc4[nH]c(Br)c(c34)C[C@H]2N(C)C1]

In some embodiments, the mental disorder or mood disorder is depression.

In some embodiments, the mental disorder or the mood disorder is major depressive disorder.

In other embodiments, the mental disorder or mood disorder is Attention Deficit Hyperactivity Disorder.

In other embodiments, the mental disorder or mood disorder is Obsessive-Compulsive Disorder.

In other embodiments, the mental disorder or mood disorder is Atypical Depression.

In other embodiments, the mental disorder or mood disorder is Psychotic major depression.

In other embodiments, the mental disorder or mood disorder is Catatonic Depression.

In other embodiments, the mental disorder or mood disorder is Postpartum Depression.

In other embodiments, the mental disorder or mood disorder is Premenstrual Dysphoric Disorder.

In other embodiments, the mental disorder or mood disorder is Seasonal Affective Disorder.

In other embodiments, the mental disorder or mood disorder is Dysthymia. In other embodiments, the mental disorder or mood disorder is Double Depression.

In other embodiments, the mental disorder or mood disorder is Depressive Disorder Not Otherwise Specified.

In other embodiments, the mental disorder or mood disorder is Depressive Personality Disorder.

In other embodiments, the mental disorder or mood disorder is Recurrent Brief Depression.

In other embodiments, the mental disorder or mood disorder is Minor Depressive Disorder.

In other embodiments, the mental disorder or mood disorder is a Bipolar Disorder (e.g. Bipolar I, Bipolar II, Cyclothymia, Bipolar disorder not otherwise specified, Impulse-Control Disorders, Adjustment Disorders, Personality Disorders).

Further in aspects, a mood disorder may include for example that involved in: bipolar and related disorders, such as bipolar I disorder, bipolar II disorder, cyclothymic disorder, substance/medication-induced bipolar and related disorder, and bipolar and related disorder due to another medical condition; substance-related disorders, such as preventing a substance use craving, diminishing a substance use craving, and/or facilitating substance use cessation or withdrawal; anxiety such as separation anxiety disorder, selective mutism, specific phobia, social anxiety disorder (social phobia), panic disorder, panic attack, agoraphobia, generalized anxiety disorder, substance/medication-induced anxiety disorder, anxiety disorder due to another medical condition; obsessive-compulsive and related disorders; trauma- and stressor-related disorders; feeding and eating disorders; neurocognitive disorders; neurodevelopmental disorders; personality disorders; sexual dysfunction; and gender dysphoria.

An effective amount of 2-Bromo-LSD or a derivative or a salt thereof, as described herein, will provide therapeutic benefit without causing substantial toxicity. The skilled artisan will appreciate that the toxicity of 2-Bromo-LSD or a derivative or a salt thereof can be determined by standard pharmaceutical procedures in cell cultures or experimental animals, for example, by determining the LD50 (the dose lethal to 50% of the population) or the LD100 (the dose lethal to 100% of the population). In some embodiments, the dose ratio between toxic and therapeutic effect is the therapeutic index. In some embodiments, the data obtained from these cell culture assays and animal studies can be used in formulating a dosage range that is not toxic for use in human. In some embodiments, the dosage of the compounds described herein lies within a range of circulating concentrations that include the effective dose with little or no toxicity. In some embodiments, the dosage may vary within this range depending upon the dosage form employed and the route of administration utilized. In some embodiments, the exact formulation, route of administration and dosage can be chosen by the individual physician in view of the patient's condition. (See, e.g., Fingl et al., 1996, In: The Pharmacological Basis of Therapeutics, 9th ed., Chapter 2, p. 29, Elliot M. Ross)

Examples of therapeutically effective doses of 2-Bromo-LSD for various mental and/or mood disorders are set forth below. In some embodiments, the term “about” when used in reference to the amount of 2-Bromo-LSD or derivative or salt thereof means about +/−1%. In some embodiments, the term “about” when used in reference to the amount of 2-Bromo-LSD or derivative or salt thereof means about +/−2%. In some embodiments, the term “about” when used in reference to the amount of 2-Bromo-LSD or derivative or salt thereof means about +/−2.5%. In some embodiments, the term “about” when used in reference to the amount of 2-Bromo-LSD or derivative or salt thereof means about +/−5%. In some embodiments, the term “about” when used in reference to the amount of 2-Bromo-LSD or derivative or salt thereof means about +/−10%. In some embodiments, the term “about” when used in reference to the amount of 2-Bromo-LSD or derivative or salt thereof means about +/−15%. In some embodiments, the term “about” when used in reference to the amount of 2-Bromo-LSD or derivative or salt thereof means about +/−20%.

| Mental and/or Mood Disorder | μg/kg/bodyweight/day |
|---|---|
| Major Depressive Disorder | about 30 |
| Attention Deficit Hyperactivity Disorder | about 15 |
| Obsessive-Compulsive Disorder | about 30 |
| Atypical Depression | about 30 |
| Melancholid Depression | about 30 |
| Psychotic major depression | about 30 |
| Catatonic Depression | about 45 |
| Postpartum Depression | about 30 |
| Premenstrual dysphoric disorder | about 25 |
| Seasonal affective disorder | about 25 |
| Dysthymia | about 30 |
| Double Depression | about 30 |
| Depressive Disorder Not Otherwise Specified | about 30 |
| Depressive personality disorder | about 30 |
| Recurrent brief depression | about 30 |
| Minor Depressive disorder | about 15 |
| Bipolar Disorders: | about 30 |
| Bipolar I | about 30 |
| Bipolar II | about 30 |
| Cyclothymia | about 30 |
| Bipolar disorder not otherwise specified | about 30 |
| Impulse-Control Disorders | about 30 |
| Adjustment Disorders | about 30 |
| Personality Disorders | about 45 |

### Combination Therapy

In some embodiments, the present disclosure relates to a method of treating or therapeutically managing a mental disorder or a mood disorder in a subject in need of treatment thereof, the method comprising administering (i) a therapeutically effective amount of 2-Bromo-LSD or a derivative or salt thereof, or a pharmaceutical composition comprising 2-Bromo-LSD or a derivative or salt thereof; and (ii) a therapeutically effective amount of an mTOR inhibitor or a pharmaceutical composition comprising the mTOR inhibitor.

In some embodiments, the methods and compositions described herein rely, in some embodiments, on blocking, reducing, or decreasing the activity of mTOR protein. In some embodiments, the inhibitors of mTOR have the ability to decrease a relevant activity of mTOR, for example, kinase activity, by about 10%, about 20%, about 30%, about 40%, about 50%, about 60%, about 70%, about 80%, about 90%, about 95% or more.

In some embodiments, the therapeutically effective amount of 2-Bromo-LSD and the therapeutically effective amount of the mTOR inhibitor are in the same unit dosage form, e.g. within the same tablet for oral administration. In some embodiments, the therapeutically effective amount of 2-Bromo-LSD and the therapeutically effective amount of the mTOR inhibitor are in different unit dosage forms, e.g. within two different tablets for oral administration. mTOR inhibitors, dosages of mTOR inhibitors, etc. are described further herein.

mTOR Inhibitors

mTOR is a large polypeptide serine/threonine kinase of the phosphatidylinositol 3-kinase (PI3K)-related kinase (PIKK) family. mTOR lies downstream from the PI3K pathway, and functions as an intermediary in a variety of cell signaling events to regulate cell growth and proliferation. mTOR activity is regulated by the serine/threonine kinase Akt, and recent evidence indicates that these kinases interact through a complex feedback inhibition pathway. mTOR modulates cell replication by controlling translation of key proteins that are required for progression of the cell cycle through the Gi to the S phase. That is, mTOR controls the translation of specific mRNAs via regulation of the phosphorylation state of several proteins involved in the translation of mRNA, mainly 4E-PB1, P7056K and eEFZ.

The mTOR pathway, with its PI3K and Akt constituents, is a critical regulator of the proliferation of cells that responds to nutrients, hormones, and growth factors, such as VEGF. Growth factors can activate PI3K signaling by binding to cognate cell surface receptors, thereby initiating a signaling cascade through Akt that results in the activation of mTOR.

mTOR is described in detail in Beugnet, et al. J Biol. Chem. 278 (42), 40717-40722 (2003); Kristof, et al., J. Biol. Chem. 278 (36), 33637-33644 (2003); Chen, Y., et al., Oncogene 22 (25), 3937-3942 (2003); Garami, et al., Mol. Cell. 11 (6), 1457-1466 (2003); Nojima, et al., J. Biol. Chem. 278 (18), 15461-15464 (2003); Kimura, et al., Genes Cells 8 (1), 65-79 (2003); McMahon, et al., Mol. Cell. Biol. 22 (21), 7428-7438 (2002); Tee, et al., Proc. Natl. Acad. Sci. U.S.A. 99 (21), 13571-13576 (2002); Hudson, et al., Mol. Cell. Biol. 22 (20), 7004-7014 (2002); Choi, et al., EMBO Rep. 3 (10), 988-994 (2002); Inoki, et al., Nat. Cell Biol. 4 (9), 648-657 (2002); Zhang, et al., J. Biol. Chem. 277 (31), 28127-28134 (2002); Castedo, et al., EMBO J. 21 (15), 4070-4080 (2002); Hara, et al., Cell 110 (2), 177-189 (2002); Kim, et al., Cell 110 (2), 163-175 (2002); Fingar, et al., Genes Dev. 16(12), 1472-1487 (2002); Reynolds, et al., J Biol. Chem. 277 (20), 17657-17662 (2002); Fang, et al., Science 294 (5548), 1942-1945 (2001); Dennis, et al., Science 294 (5544), 1102-1105 (2001); Onyango, et al., Genomics 50 (2), 187-198 (1998); Lench, et al., Hum. Genet. 99 (4), 547-549 (1997); Choi, et al., Science 273 (5272), 239-242 (1996); Moore, et al., Genomics 33 (2), 331-332 (1996); Chen, et al., Proc. Natl. Acad. Sci. U.S.A. 92 (11), 4947-4951 (1995); Chiu et al., Proc. Natl. Acad. Sci. U.S.A. 91 (26), 12574-12578 (1994); Brown, et al., Nature 369 (6483), 756-758 (1994).

In some embodiments of the invention, the mTOR inhibitor may be any one or more of a small molecule, a peptide, an antibody or a fragment thereof, a nucleic acid molecule and/or a macrolide compound. In some embodiments, the antibody specifically binds mTOR so as to inhibit mTOR. In some embodiments, the antibody may be any one or more of a monoclonal antibody or fragment thereof, a polyclonal antibody or a fragment thereof, a chimeric antibody, a humanized antibody, a human antibody, or a single chain antibody. In some embodiment, these antibodies can be from any source, e.g., rat, dog, cat, pig, horse, mouse or human. In some embodiment, fragments of antibodies may be any one or more of Fab, F(ab′)2, Fv fragments or fusion proteins.

Anti-peptide antibodies and other biological molecules useful in reducing the activity of mTOR or which function as mTOR inhibitors are disclosed in U.S. Pat. Nos. 9,249,462 and 8,840,899, the disclosures of which are hereby incorporated by reference herein in their entireties. For example, antisense compounds that specifically target and down-regulate expression of mTOR can be used with the methods provided herein. In some embodiments, antisense compounds specifically targeting mTOR can be prepared by designing compounds that are complementary to an mTOR nucleotide sequence, particularly the mTOR mRNA sequence. Antisense compounds targeting mTOR need not be 100% complementary to mTOR to specifically hybridize and regulate expression the target gene. For example, the antisense compound, or antisense strand of the compound. If a double-stranded compound, can be at least 75%, at least 80%, at least 85%, at least 90%, at least 95%, at least 99% or 100% complementary to the selected mTOR nucleic acid sequence. Methods of screening antisense compounds for specificity are well known in the art (see, for example, U.S. Patent Application Publication No. 2003-0228689). Exemplary mTOR shRNA sequences are provided herein as SEQ ID NOs:10 and 11.

In some embodiments, the mTOR inhibitors are small molecules. In some embodiments, the small molecule mTOR inhibitors include rapamycin (sirolimus) and derivatives, analogs, and prodrugs thereof. Rapamycin is a 31-membered macrolide lactone, C₅₁H₇₉NO₁₃, having a molecular mass of 913.6 Da. In solution, sirolimus forms two conformational trans-, cis-isomers with a ratio of 4:1 (chloroform) due to hindered rotation around the pipecolic acid amide bond. Rapamycin is sparingly soluble in water, aliphatic hydrocarbons, and diethyl ether, whereas it is soluble in alcohols, halogenated hydrocarbons and dimethyl sulfoxide. It is believed that rapamycin is unstable in solution and degrades in plasma and low, and neutral-pH buffers at 37 C with half-life of less than about 10 hours.

The structure of rapamycin is disclosed in U.S. Pat. No. 8,685,995, the disclosure of which is hereby incorporated by reference herein in its entirety. Methods for the preparation of rapamycin are disclosed in Sehgal et al., U.S. Pat. Nos. 3,929,992, and 3,993,749. In addition, monoacyl and diacyl derivatives of rapamycin and methods for their preparation are disclosed by Rakhit, U.S. Pat. No. 4,316,885. Furthermore, Stella et al., U.S. Pat. No. 4,650,803 disclose water soluble prodrugs of rapamycin, i.e., rapamycin derivatives including the following rapamycin prodrugs: glycinate prodrugs, propionate prodrugs and the pyrrolidino butyrate prodrugs (“prodrugs” are described further herein).

The methods and compositions described herein include the use of natural and synthetic rapamycin, genetically engineered rapamycin and all derivatives and prodrugs of rapamycin, such as described in the aforementioned U.S. Pat. Nos. 3,929,992; 3,993,749; 4,316,885; and 4,650,803, the contents of which are hereby incorporated by reference.

Rapamycins, as that term is used herein, includes rapamycin itself, and esters, ethers, carbamates, oximes, hydrazones, and hydroxylamines of rapamycin; as well as rapamycins in which functional groups on the rapamycin nucleus have been modified, for example through reduction or oxidation. Also included are oximes, hydrazones, and hydroxylamines of rapamycin as disclosed in U.S. Pat. Nos. 5,373,014, 5,378,836, 5,023,264, and 5,563,145. The preparation of these oximes, hydrazones, and hydroxylamines is disclosed in the above-listed patents. The preparation of 40-oxorapamycin is disclosed in U.S. Pat. No. 5,023,263. Each of these patents are hereby incorporated by reference herein in their entireties.

In some embodiments, the mTOR inhibitor is a derivative of rapamycin. In some embodiments, rapamycin derivatives have the basic rapamycin structure with substitutions at the C-40 position. If the substituent at position 40 is designated as R, then the following substitutions and corresponding compounds are: R=—OP(O)(Me)₂, AP23573 (WO1998/02441 and WO 2001/14387); R=—OC(O)C(CH₃)(CH₂OH), temsirolimus (U.S. Pat. No. 5,362,718); R=—OCH₂CH₂OH, everolimus (U.S. Pat. No. 5,665,772); R=OCH₂CH₂OEt, biolimus; R=-tetrazole, ABT-578 (WO 1999/15530). Each of these patents are hereby incorporated by reference herein in their entireties.

In other embodiments, rapamycin derivatives include substitutions in the C-40 and/or C-16 and/or C-32 positions. Esters and ethers of rapamycin are described in the following patents, which are all hereby incorporated by reference herein in their entireties: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. Nos. 5,118,678; 5,411,967; 5,434,260; 5,480,988; 5,480,989; 5,489,680); silyl esters (U.S. Pat. No. 5,120,842); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); amidino carbamate esters (U.S. Pat. No. 5,463,048); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462); 32-esters and ethers (U.S. Pat. No. 5,256,790). The synthesis of these esters and ethers is disclosed in the patents listed above. Each of these patents are hereby incorporated by reference herein in their entireties.

Yet other derivatives have one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and elimination, derivatization or replacement of one or more substituents of the cyclohexyl ring or replacement of the cyclohexyl ring with a substituted or unsubstituted cyclopentyl ring.

In some embodiments, the mTOR inhibitor may be provided in the form of a prodrug. The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375 382,615th Meeting Belfast (1986) and Stella et al., “Prodrugs: A Chemical Approach to Targeted Drug Delivery,” Directed Drug Delivery, Borchardt et al., (ed.), pp. 247 267, Humana Press (1985). The prodrugs described here include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of mTOR inhibitors that can be derivatized into a prodrug form for use in the methods and compositions described here include, but are not limited to, those mTOR inhibitors agents described above. By way of example, Temsirolimus, described herein, is a prodrug of rapamycin.

Specific derivatives of rapamycin which may be used in the methods and compositions described herein include RAD001 (Everolimus) and CCI 779 (Temsirolimus). Everolimus has the molecular formula C₅₃H₈₃NO₁₄ and has the molecular mass 958.224 g/mol. Everolimus is identified by its CAS number 159351-69-6, ATC code L04AA18 and PubChem 6442177. Everolimus and is manufactured by Novartis AG. Everolimus is currently used as an immunosuppressant to prevent rejection of organ transplants. Everolimus is described in detail in O'Reilly T M, Wood J M, Littlewood-Evans A, et al. Differential anti-vascular effects of mTOR or VEGFR pathway inhibition: a rational basis for combining RAD001 and PTK787/ZK222584. Presented at: 96th Annual Meeting of the American Association for Cancer Research. Anaheim, Calif.; Apr. 16-20, 2005. Abstract 3038. Everolimus is also described in Van Oosterom A T, Dumez H, Desai J, et al. Combination signal transduction inhibition: a phase I/II trial of the oral mTOR-inhibitor everolimus (E, RAD001) and imatinib mesylate (IM) in patients (pts) with gastrointestinal stromal tumor (GIST) refractory to IM [abstract]. Proc Am Soc Clin Oncol. 2004; 23:195. Abstract 3002.

Temsirolimus (cell cycle inhibitor-779, C56H87NO16, molecular weight 1030.3) is an ester analogue of Rapamycin. Temsirolimus is also known as rapamcyin-28-N,N-dimethlyglycinate methanesulfonate salt, rapamycin, 42-[3-hydroxy-2-(hydroxymethyl)-2-methylpropanoate], (3S,6R,7E,9R,10R,12R,14S,15E,17E,19E,21S,23S,26R,27R,34aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4] oxaazacyclohentriacontine-1,5,11,28,29(4H,6H,31H)-pentone 4′-[2,2-bis(hydroxymethyl)propionate] and rapamycin 42-[2,2-bis(hydroxymethyl)propionate]. Temsirolimus is described in detail in Nat. Genet. 2004; 36:585-95 and J Clin Oncol. 2004; 22:2336-47. Reference should also be made to K Yu, et al., (2001). mTOR, a novel target in breast cancer: the effect of CCI-779, an mTOR inhibitor, in preclinical models of breast cancer. Endocrine-Related Cancer 8 (3) 249-258 and Josep Maria Peralba, Linda deGraffenried, William Friedrichs, Letitia Fulcher, Viktor Grunwald, Geoffrey Weiss and Manuel Hidalgo (2003). Pharmacodynamic Evaluation of CCI-779, an Inhibitor of mTOR, in Cancer Patients. Clinical Cancer Research Vol. 9, 2887-2892.

Other small molecule inhibitors of mTOR include fused bicyclic compounds (WO 2007/61737, WO 2007/87395 and WO 2007/64993), heteroaromatic amines (WO 2001/19828), pyrrolopyrimidine compounds (WO 2005/47289), diphenyl-dihydro-indol-2-one derivatives (WO 2005/97107), and trimethy-dodeca-triene derivatives (US Patent Publication No. 2007/037887). Additional mTOR inhibitors, including rapamycin derivatives and analogs have been described, such as, for example, those disclosed in WO2007/135411, WO1998/02441, WO2001/14387 and WO2003/64383; and EP1880723. Each of these patents are hereby incorporated by reference herein in their entireties.

Compositions for transdermal delivery of mTOR inhibitors are disclosed in U.S. Pat. No. 10,172,789, the disclosure of which is hereby incorporated by reference herein in its entirety.

Combination pharmaceutical compositions and uses thereof are disclosed in U.S. Pat. No. 10,172,858, the disclosure of which is hereby incorporated by reference herein in its entirety.

mTOR Inhibitor Dosage and Dosage Forms

In embodiments of the invention, an mTOR inhibitor compound may be administered as appropriate including by any administration route, for example, enterally, (e.g., orally), or parenterally or topically. In some embodiments, the mTOR inhibitor and 2-Bromo-LSD may be administrated via the same route of administration, e.g. both orally. In other embodiments, the mTOR inhibitor and 2-Bromo-LSD may be administrated via the differing routes of administration, e.g. one orally and another intravenously.

In some embodiments, the mTOR inhibitor is rapamycin or a rapamycin analog. In one embodiment, the dose of rapamycin or the rapamycin analog is about 0.2 to about 1.0 mg/kg, such as about 0.4 to about 0.8 mg/kg. In some examples, the dose of rapamycin or the rapamycin analog is about 0.2, about 0.3, about 0.4, about 0.5, about 0.6, about 0.7, about 0.8, about 0.0 or about 1.0 mg/kg. In another embodiment, the dose of rapamycin is a low dose of rapamycin. In one embodiment, the low dose of rapamycin is about 0.01 to about 0.15 mg/kg, such as about 0.05 to about 0.1 mg/kg. In some examples, a low dose of rapamycin is about 0.01, about 0.02, about 0.03, about 0.04, about 0.05, about 0.06, about 0.07, about 0.08, about 0.09, about 0.10, about 0.11, about 0.12, about 0.13, about 0.14 or about 0.15 mg/kg. In this context, “about” refers to a value within 0.005 mg/kg.

In some embodiments, the effective amounts of the mTOR inhibitors for use with the claimed methods and/or in the pharmaceutical compositions be in the range of about 0.1 to about 0.5 mg/day, about 0.5 to about 1.0 mg/day, about 1.0 to about 1.5 mg/day, about 1.5 to about 2 mg/day, about 2.0 to about 2.5 mg/day, about 2.5 to about 5 mg/day, about 5 to about 10 mg/day, about 10 to about 15 mg/day, about 15 to about 20 mg/day, about 20 about 25 mg/day, about 25 about 30 mg/day, about 30 about 35 mg/day, about 35 about 40 mg/day, about 40 about 45 mg/day, about 45 about 50 mg/day, about 50 to about 55 mg/day, about 55 to about 60 mg/day, about 60 to about 65 mg/day, about 65 to about 70 mg/day, about 70 to about 75 mg/day, about 75 to about 80 mg/day, about 80 to about 85 mg/day, about 85 to about 90 mg/day, about 90 to about 95 mg/day, about 95 to about 100 mg/day, about 0.75 to about 10 mg/day or about 2 to about 10 mg/day. In some embodiments of the invention, the mTOR inhibitors are Temsirolimus (CCI to 779) or a pharmaceutical equivalent, analog, derivative, or a salt thereof, Evirolimus (RAD001) or a pharmaceutical equivalent, analog, derivative, or a salt thereof, and/or Rapamycin or a pharmaceutical equivalent, analog, derivative or a salt thereof.

In alternate embodiments, the effective amounts of the mTOR inhibitors in the composition for use with the claimed methods, in the pharmaceutical compositions and/or in the claimed kits may be in the range of about 1 to about 5 mg/week, about 5 to about 10 mg/week, about 10 to about 15 mg/week, about 15 to about 20 mg/week, about 20 to about 25 mg/week, about 25 to about 30 mg/week, about 30 to about 35 mg/week, about 35 to about 40 mg/week, about 40 to about 45 mg/week, about 45 to about 50 mg/week, about 50 to about 55 mg/week, about 55 to about 60 mg/week, about 60 to about 65 mg/week, about 65 to about 70 mg/week, about 70 to about 75 mg/day, about 75 to about 80 mg/mg, about 80 to about 85 mg/mg, about 85 to about 90 mg/week, about 90 to about 95 mg/week or about 95 to about 100 mg/week. In some embodiments of the invention, the mTOR inhibitors are Temsirolimus (CCI-779) or a pharmaceutical equivalent, analog, derivative, or a salt thereof, Evirolimus (RAD001) or a pharmaceutical equivalent, analog, derivative or a salt thereof, and/or Rapamycin or a pharmaceutical equivalent, analog, derivative or a salt thereof.

For example, in some embodiments, Everolimus may be administered orally, in daily dosages from about 0.1 mg up to about 25 mg or about 0.1 mg to about 15 mg, including about 0.1 mg, about 0.25 mg, about 0.5 mg, about 0.75 mg, about 1 mg, about 2.5 mg, about 5 mg, or about 10 mg, e.g., in the form of dispersible tablets or in the form of a solid dispersion, depending on the disease being treated. In some embodiments, Everolimus may be administered in a weekly dosage that may include up to about 70 mg, such as about 10 to about 70 mg, or about 30 to about 50 mg, depending on the disease being treated. For further example, tacrolimus (Protopic) may be administered as an ointment of about 0.03% to about 0.1% (w/w) in an ointment base. Other mTOR inhibitors may be administered analogously, e.g. in similar dosage ranges.

For parental administration, including intravenous administration, an initial intravenous dosage may range from between about 0.1 and about 100 mg/m² when administered on a daily dosage regimen (daily for five days, every two to three weeks), and more suitably, between about 0.1 and about 1000 mg/m² when administered on a once weekly dosage regimen.

In some embodiments, the mTOR inhibitor is administered prior to administration of the 2-Bromo-LSD. In some embodiments, the mTOR inhibitor is administered concurrently with the administration of the 2-Bromo-LSD. In some embodiments, the mTOR inhibitor is administered sequentially with the administration of the 2-Bromo-LSD. In some embodiments, the mTOR inhibitor is administered after administration of the 2-Bromo-LSD.

In some embodiment, the mTOR inhibitor is administered in a single dose. In other embodiments, the mTOR inhibitor is administered in multiple doses. When administered in multiple doses, the dosing schedule of the mTOR inhibitor can vary. In some embodiments, the mTOR inhibitor is administered twice a day, daily, weekly, or monthly. In some embodiments, the mTOR inhibitor is administered daily for about one week. In other embodiments, the mTOR inhibitor is administered daily for about one month.

In some examples, the mTOR inhibitor is administered in 1 to about 40 doses, such as about 5 to about 30 doses, about 10 to about 25 doses, or about 15 to about 20 doses. When administered in multiple doses, the mTOR inhibitor can be administered prior to, on the same day as, or following administration of 2-Bromo-LSD, or a combination thereof. For example, a subject can be administered the mTOR inhibitor daily for three days prior to administration of 2-Bromo-LSD. As another example, a subject can be administered the mTOR inhibitor on the same day as 2-Bromo-LSD and then administered the mTOR inhibitor daily for up to one week. In some embodiments, the mTOR inhibitor is administered daily. In some examples, the mTOR inhibitor is administered daily for one week. In other embodiments, the mTOR inhibitor is administered weekly. In some embodiments, the mTOR inhibitor is administered continuously, such as part of a patch or other transdermal delivery means.

The mTOR inhibitors can be administered by any suitable route. The route of administration will be determined by a variety of factors, including the type of inhibitor used, and the composition of inhibitor (e.g., liquid or solid form). Methods of administration include, but are not limited to, intradermal, topical, intramuscular, transdermal, intraperitoneal, parenteral, intravenous, subcutaneous, vaginal, rectal, intranasal, inhalation, oral or mist-spray delivery to the lungs. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local. Sterile injectable solutions are prepared by incorporating the active compounds in the required amount in the appropriate solvent with any other ingredients as required, followed by filtered sterilization.

In some embodiments, the mTOR inhibitor is administered topically or transdermally, for example in a patch, pad, bandage, cream, gel, lotion, spray, foam or paste. When administered as a patch, pad, bandage or the like, the patch, pad or bandage can be replaced at regular intervals to maintain a constant dose of mTOR inhibitor. Alternatively, the patch, pad or bandage can be applied for a given time period, such as one day, two days, three days, four days, five days, six days or seven days, or until the mTOR inhibitor is depleted from the patch, pad or bandage. Patches suitable for transdermal delivery of therapeutic agents are known in the art (see, for example, U.S. Patent Application Publication Nos. 2005/0142176; 2008/0274166; 2009/0028929; and 2009/0048567).

### Pharmaceutically Acceptable Carriers

Pharmaceutically acceptable carriers include diluents and excipients generally used in pharmaceutical preparations, such as fillers, extenders, binders, moisturizers, disintegrators, surfactant, lubricants, etc. Non-limiting examples of suitable carriers are described herein.

### Diluents

A diluent may be selected from, for example, calcium carbonate, calcium phosphate dibasic, calcium phosphate tribasic, calcium sulfate, microcrystalline cellulose, microcrystalline silicified cellulose, powdered cellulose, dextrate, dextrose, fructose, lactitol, lactose anhydrous, lactose monohydrate, lactose dihydrate, lactose trihydrate, mannitol, sorbitol, starch, pregelatinized starch, sucrose, talc, xylitol, maltose, maltodextrin, maltitol. In some embodiments, the diluent is selected from starches, lactose, cellulose derivatives, confectioner's sugar and the like. Different grades of lactose include, but are not limited to, lactose monohydrate, lactose DT (direct tableting), lactose anhydrous, and others. Different starches include, but are not limited to, maize starch, potato starch, rice starch, wheat starch, pregelatinized starch, and others. Different celluloses that can be used include crystalline celluloses, such as a microcrystalline cellulose, and powdered celluloses. Other useful diluents include, but are not limited to, carmellose, sugar alcohols such as mannitol, sorbitol, and xylitol, calcium carbonate, magnesium carbonate, dibasic calcium phosphate, and tribasic calcium phosphate.

### Binders

A binder may be selected from, for example, acacia, alginic acid, carbomer, carboxymethylcellulose calcium, carbomethylcellulose sodium, microcrystalline cellulose, powdered cellulose, ethyl cellulose, gelatin liquid glucose, guar gum, hydroxyethyl cellulose, hydroxypropyl cellulose, hydroxypropylmethyl cellulose, maltodextrin, methylcellulose, polydextrose, polyethtylene oxide, povidone, sodium alginate, starch paste, pregelatinized starch, sucrose, tragacanth, low-substituted hydroxypropyl cellulose, glucose, sorbitol.

### Fillers

A suitable filler may be selected from, for example, starch derivatives, such as corn starch, potato starch or rice starch, polysaccharides such as dextrins, maltodextrins, dextrates, microcrystalline cellulose, powdered cellulose, mixture of microcrystalline cellulose and guar gum, coprocessed blends of microcrystalline cellulose; and polyhydric alcohols, such as xylitol and sorbitol.

### Disintegrants

A disintegrant may be selected from, for example, alginic acid, carbon dioxide, carboxymethylcellulose calcium, carboxymethylcellulose sodium, microcrystalline cellulose, powdered cellulose, croscarmelose sodium, crospovidone, sodium docusate, gaur gum, hydroxypropyl cellulose, methylcellulose, polacrilin potassium, poloxamer, povidone, sodium alginate, sodium glycine carbonate, sodium lauryl sulfate, sodium starch glycolate, starch, pregelatinized starch, low-substituted hydroxypropyl cellulose.

### Glidants

A glidant may be selected from, for example, calcium silicate, powdered cellulose, starch, talc, colloidal silicon dioxide.

### Lubricants

A lubricant may be selected from, for example, magnesium stearate, stearic acid, sodium stearyl fumarate, magnesium lauryl sulphate, talc, polyethylene glycol, and glyceryl behenate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and any combinations thereof.

The pharmaceutical composition of the present disclosure may be formulated as an ordinary pharmaceutical preparation, for example in the form of tablets, flash melt tablets, pills, powder, liquid, suspension, emulsion, granules, capsules, suppositories or injection (liquid, suspension, etc.), troches, intranasal spray percutaneous patch and the like.

### Absorption Enhancers

Absorption enhancers for use in accordance with certain embodiments of the present disclosure include, for example, Gelucire 44/14; Gelucire 50/13; Tagat TO; Tween 80; isopropyl myristate, polysorbates, sorbitan esters, poloxamer block copolymers, PEG-35 castor oil, PEG-40 hydrogenated castor oil, caprylocaproyl macrogol-8 glycerides, PEG-8 caprylic/capric glycerides, sodium lauryl sulfate, dioctyl sulfosuccinate, polyethylene lauryl ether, ethoxydiglycol, propylene glycol mono-di-caprylate, glycerol monocaprylate, glyceryl fatty acids (C8-C18) ethoxylated, oleic acid, linoleic acid, glyceryl caprylate/caprate, glyceryl monooleate, glyceryl monolaurate, caprylic/capric triglycerides, ethoxylated nonylphenols, PEG-(8-50) stearates, olive oil PEG-6 esters, triolein PEG-6 esters, lecithin, d-alpha tocopheryl polyethylene glycol 1000 succinate, polycarbonate, sodium glycocholate, sodium taurocholate, cyclodextrins, citric acid, sodium citrate, triacetin, combinations thereof, and the like. In certain preferred embodiments, the absorption enhancer is triacetin.

### Sweeteners/Flavoring Agents

A suitable sweetener may be selected from sugars such as sucrose, lactose and glucose; cyclamate and salts thereof; saccharin and salts thereof; and aspartame.

Flavoring agents may be incorporated in the composition may be chosen from synthetic flavors oils and flavoring aromatics, natural oils, plant extracts. Examples include cinnamon oil, oil of wintergreen, peppermint oil, clove oil, bay oil, anise oil, eucalyptus, thyme oil, cedar leaf oil, nutmeg oil, sage oil or almond oil. Examples of flavoring agents include, but are not limited to, almond, apple, banana, berry, bubblegum, caramel, citrus, cherry, chocolate, coconut, grape, green tea, honey, lemon, licorice, lime, mango, maple, mint, orange, peach, pineapple, raisin, strawberry, vanilla, watermelon and combinations thereof. Flavors may be present in an amount ranging from about 0.001001% to about 5% by total weight of the formulation. In some embodiments, the flavoring agent may be selected from natural or synthetic flavors such as, for example, strawberry flavor, wild cherry flavor, green apple flavor, spearmint flavor and peppermint flavor. In some embodiments, the flavoring agents are selected from menthol, peppermint, wintergreen, orange, cherry, and other fruits, vanilla, almond and other nuts, etc.

In some embodiments the pharmaceutical compositions of the present disclosure are in the form of tablets, which may include one or more pharmaceutically acceptable carriers or excipients selected from lactose, saccharose, sodium chloride, glucose, urea, starch, xylitol, mannitol, erythritol, sorbitol, calcium carbonate, kaolin, crystalline cellulose, silic acid and other excipients; water, ethanol, propanol, simple syrup, glucose solution, starch solution, gelatin solution, carboxymethyl cellulose, shellac, methyl cellulose, potassium phosphate, polyvinyl pyrrolidone and other binders; dried starch, sodium alginate, agar powder, laminaran powder, sodium hydrogencarbonate, calcium carbonate, polyoxyethylene sorbitan fatty acid esters, sodium lauryl sulfate, stearic acid monoglyceride, starch, lactose and other disintegrators; white sugar, stearin, cacao butter, hydrogenated oil and other disintegration inhibitors; quaternary ammonium salt, sodium lauryl sulfate and other absorption accelerator; glycerine, starch and other moisture retainers; starch, lactose, kaolin, bentonite, colloidal silic acid and other adsorbents; and refined talc, stearate, boric acid powder, polyethylene glycol and other lubricants and the like. Tablets can also be formulated with ordinary coatings, such as sugar-coated tablets, gelatin-coated tablets, enteric coated tablets and film coated tablets, as well as double tablets and multilayered tablets.

In some embodiments the pharmaceutical compositions of the present disclosure are in the form of pills, which may include one or more pharmaceutically acceptable carriers or excipients selected from glucose, lactose, starch, cacao butter, hardened vegetable oil, kaolin, talc and other excipients; gum arabic powder, traganth powder, gelatin, ethanol and other binders; and laminaran, agar and other disintegrators and the like.

In some embodiments the pharmaceutical compositions of the present disclosure are in the form of capsules. Capsules are prepared according to ordinary methods by mixing carbostyril derivatives such as anhydrous aripiprazole crystals as the first ingredient and serotonin reuptake inhibitor as the second ingredient, and the various carriers described above and packing them in hard gelatin capsules, soft capsules hydroxypropylmethyl cellulose capsules (HPMC capsules) and the like.

In some embodiments the pharmaceutical compositions of the present disclosure are in the form of suppositories, which may include one or more pharmaceutically acceptable carriers or excipients selected from polyethylene glycol, cacao butter, higher alcohol, esters of higher alcohol, gelatin semi-synthetic glyceride and the like.

### Routes of Administration and Dosage Forms

Administration to a subject of the formulations according to the present disclosure may be via any common route so long as the target tissue is available via that route. The formulations may conveniently be presented in dosage unit form and may be prepared by any of the methods well known in the art of pharmacy. In some embodiments, the formulations are prepared by uniformly and intimately bringing the active components into association with a liquid carrier or a finely divided solid carrier or both, and then, if necessary, shaping the product into the desired dosage form. Of course, the skilled artisan will recognize that the active components (e.g. 2-Bromo-LSD) are included in an amount sufficient to produce the desired pharmacologic effect.

In some embodiments, the composition is administered depending on the type of preparation form, and the age, gender and other condition of the patient (degree and conditions of the disease, etc.). For example, tablets, pills, liquids, suspensions, emulsions, granules and capsules are administered orally. In case of an injectable preparation, it is administered intravenously by either singly or mixed with a common auxiliary liquid such as solutions of glucose or amino acid. Further, if necessary, the injectable preparation is singly administered intracutaneously, subcutaneously or intraperitoneally. In case of a suppository, it is administered intrarectally.

In some embodiments, 2-Bromo-LSD or a derivative or a salt thereof is administered at a dosage, such as described herein, at least once a day. In some embodiments, 2-Bromo-LSD or a derivative or a salt thereof is administered at a dosage, such as described herein, at least twice a day. In some embodiments, 2-Bromo-LSD or a derivative or a salt thereof is administered at a dosage, such as described herein, at least three times a day.

In other embodiments, 2-Bromo-LSD or a derivative or a salt thereof is administered at a dosage, such as described herein, at least once every other day. In yet other embodiments, 2-Bromo-LSD or a derivative or a salt thereof is administered at a dosage, such as described herein, at least once every third day. In further embodiments, 2-Bromo-LSD or a derivative or a salt thereof is administered at a dosage, such as described herein, at least once every fourth day. In further embodiments, 2-Bromo-LSD or a derivative or a salt thereof is administered at a dosage, such as described herein, or at least once every fifth day.

In some embodiments, the methods and formulations can be practiced as a single, one time dose or chronically. By chronic it is meant that the methods and compositions of the disclosure are practiced more than once to a given subject or individual. For example, chronic administration can be multiple doses of a pharmaceutical composition administered to a subject, on a daily basis, a weekly basis, a biweekly basis, monthly basis, or more or less frequently, as will be apparent to those of skill in the art. Chronic administration can continue for weeks, months, or years if appropriate according to the judgment of the practitioner of skill in the art. Furthermore, if certain doses, in the judgment of the practitioner of skill in the art, show tolerability profiles which may not be acceptable, the practitioner can reduce the dose to reduce such profiles.

### Aspects of the Invention:

1. A method of treating major depressive disorder comprising administering to a subject a therapeutically effective amount of 2-Bromo-LSD or a derivative or salt thereof.
2. The method of claim 1, wherein the therapeutically effective amount of the 2-Bromo-LSD is administered at least once per day.
3. The method of claim 1 or 2, wherein the therapeutically effective amount is up to 50 μg or about 50 μg of said 2-Bromo-LSD or the derivative or salt thereof.
4. The method of claim 3, wherein the therapeutically effective amount is about 27 μg to about 33 μg of said 2-Bromo-LSD or the derivative or salt thereof.
5. The method of claim 4, wherein the therapeutically effective amount is about 30 μg of said 2-Bromo-LSD or the derivative or salt thereof.
6. The method of any one of claims 1 to 5, wherein the 2-Bromo-LSD or the derivative or salt thereof is formulated as a pharmaceutical composition further comprising a pharmaceutically acceptable carrier or excipient.
7. The method of claim 6, wherein the pharmaceutical composition is formulated for intravenous, intramuscular, intraperitoneal, oral, or inhalation administration.
8. The method of claim 7, wherein the pharmaceutical composition is formulated as a tablet or as a capsule.
9. The method of claim 6 or 7, wherein the pharmaceutical composition is formulated as an oral dosing solution.
10. The method of any one of claims 1 to 9, further comprising the administration of a mTOR inhibitor prior to or with said 2-Bromo-LSD or a derivative or salt thereof, optionally wherein said mTOR inhibitor is formulated in a same or a different formulation format to said 2-Bromo-LSD.
11. A method of treating a mental disorder and/or a mood disorder comprising administering to a subject a therapeutically effective amount of 2-Bromo-LSD or a derivative or salt thereof,
   wherein the mental disorder and/or the mood disorder is selected from the group consisting of Attention Deficit Hyperactivity Disorder, Obsessive-Compulsive Disorder, Atypical Depression, Melancholid Depression, Psychotic major depression, Catatonic Depression, Postpartum Depression, Premenstrual dysphoric disorder, Seasonal affective disorder, Dysthymia Double Depression, Depressive Disorder Not Otherwise Specified, Depressive personality disorder, Recurrent brief depression, and Minor Depressive disorder.
12. The method of claim 11, wherein the therapeutically effective amount of the 2-Bromo-LSD is administered at least once per day.
13. The method of claim 11 or 12, wherein the therapeutically effective amount is up to about 35 μg of said 2-Bromo-LSD or the derivative or salt thereof.
14. The method of claim 13, wherein the therapeutically effective amount is about 15 μg to about 30 μg of said 2-Bromo-LSD or the derivative or salt thereof.
15. The method of claim 14, wherein the therapeutically effective amount is about 30 μg of said 2-Bromo-LSD or the derivative or salt thereof.
16. The method of any one of claims 11 to 15, wherein the 2-Bromo-LSD or a derivative or salt thereof is formulated as a pharmaceutical composition further comprising a pharmaceutically acceptable carrier or excipient.
17. The method of claim 16, wherein the pharmaceutical composition is formulated for intravenous, intramuscular, intraperitoneal, oral, or inhalation administration.
18. The method of claim 17, wherein the pharmaceutical composition is formulated as a tablet or as a capsule.
19. The method of claim 16 or 17, wherein the pharmaceutical composition is formulated as an oral dosing solution.
20. The method of any one of claims 11 to 19, further comprising the administration of a mTOR inhibitor prior to, concurrently with, or within set times with said 2-Bromo-LSD or the derivative or salt thereof, optionally wherein said mTOR inhibitor is formulated as the same or a different formulation format to said 2-Bromo-LSD.
21. A method of treating a bipolar disorder comprising administering to a subject a therapeutically effective amount of 2-Bromo-LSD or a derivative or salt thereof.
22. The method of claim 21, wherein the therapeutically effective amount of the 2-Bromo-LSD is administered at least once per day.
23. The method of claim 21 or 22, wherein the therapeutically effective amount is up to about 35 μg of said 2-Bromo-LSD or the derivative or salt thereof.
24. The method of claim 23, wherein the therapeutically effective amount is about 27 μg to about 33 μg of said 2-Bromo-LSD or the derivative or salt thereof.
25. The method of claim 24, wherein the therapeutically effective amount is about 30 μg of said 2-Bromo-LSD or the derivative or salt thereof.
26. The method of any one of claims 21 to 25, wherein the 2-Bromo-LSD or the derivative or salt thereof is formulated as a pharmaceutical composition further comprising a pharmaceutically acceptable carrier or excipient.
27. The method of claim 26, wherein the pharmaceutical composition is formulated for intravenous, intramuscular, intraperitoneal, oral, or inhalation administration.
28. The method of claim 27, wherein the pharmaceutical composition is formulated as a tablet or as a capsule.
29. The method of claim 16 or 17, wherein the pharmaceutical composition is formulated as an oral dosing solution.
30. The method of any one of claims 21 to 29, further comprising the administration of a mTOR inhibitor prior to, concurrently with, or within set times with said 2-Bromo-LSD or the derivative or salt thereof.
31. The method of claim 30, wherein said mTOR inhibitor is formulated as the same or in a different formulation format to said 2-Bromo-LSD.
32. The method of any one of claims 21 to 31, wherein the bipolar disorder is selected from the group consisting of bipolar I, bipolar II, cyclothymia, and bipolar disorder not otherwise specified.
33. A method of treating a mental disorder and/or a mood disorder comprising administering to a subject a therapeutically effective amount of 2-Bromo-LSD or a derivative or salt thereof, wherein the mental disorder and/or the mood disorder is selected from the group consisting of impulse-control disorders, adjustment disorders, and personality disorders.
34. The method of claim 33, wherein the therapeutically effective amount of the 2-Bromo-LSD is administered at least once per day.
35. The method of claim 33 or 34, wherein the therapeutically effective amount is up to about 50 μg of said 2-Bromo-LSD or the derivative or salt thereof.
36. The method of claim 35, wherein the therapeutically effective amount is about 25 μg to about 50 μg of said 2-Bromo-LSD or the derivative or salt thereof.
37. The method of claim 36, wherein the therapeutically effective amount is about 30 μg to about 45 μg of said 2-Bromo-LSD or the derivative or salt thereof.
38. The method of any one of claims 33 to 37, wherein the 2-Bromo-LSD or a derivative or salt thereof is formulated as a pharmaceutical composition further comprising a pharmaceutically acceptable carrier or excipient.
39. The method of claim 38, wherein the pharmaceutical composition is formulated for intravenous, intramuscular, intraperitoneal, oral, or inhalation administration.
40. The method of claim 39, wherein the pharmaceutical composition is formulated as a tablet or as a capsule.
41. The method of claim 38 or 39, wherein the pharmaceutical composition is formulated as an oral dosing solution.
42. The method of any one of claims 33 to 41, further comprising the administration of a mTOR inhibitor prior to, concurrently with, or within set times with said 2-Bromo-LSD or the derivative or salt thereof, optionally wherein said mTOR inhibitor is formulated as the same or a different formulation format to said 2-Bromo-LSD.
43. A method of treating major depressive disorder in a subject in need thereof, wherein the subject has been diagnosed with the major depressive disorder, comprising: administering a therapeutically effective amount of a mTOR inhibitor and a therapeutically effective amount of 2-Bromo-LSD to the subject, wherein the mTOR inhibitor is selected from the group consisting of (i) temsirolimus (CCI-779) or a pharmaceutical equivalent, analog, derivative or a salt thereof, (ii) Everolimus (RAD001) or a pharmaceutical equivalent, analog, derivative or a salt thereof, and (iii) Rapamycin or a pharmaceutical equivalent, analog, derivative or a salt thereof.
44. The method of claim 43, wherein the mTOR inhibitor and the 2-Bromo-LSD are administered concurrently.
45. The method of claim 43, wherein the mTOR inhibitor and the 2-Bromo-LSD are administered sequentially.
46. The method of claim 43, wherein the mTOR inhibitor and the 2-Bromo-LSD are administered within 12 hours of each other.
47. The method of claim 43, wherein the mTOR inhibitor and the 2-Bromo-LSD are administered within 6 hours of each other.
48. The method of claim 43, wherein the mTOR inhibitor and the 2-Bromo-LSD are administered within 4 hours of each other.
49. The method of claim 43, wherein the mTOR inhibitor and the 2-Bromo-LSD are administered within 2 hours of each other.
50. The method of any one of claims 43 to 49, wherein the effective amount of the mTOR inhibitor is 0.1-0.5 mg/day. 0.5-1.0 mg/day, 1.0-1.5 mg/day, 1.5-2 mg/day, 2.0-2.5 mg/day, 2.5-5 mg/day, 5-10 mg/day, 10-15 mg/day, 15-20 mg/day, 20-25 mg/day, 25-30 mg/day, 30-35 mg/day, 35-40 mg/day, 40-45 mg/day, 45-50 mg/day, 50-55 mg/day, 55-60 mg/day, 60-65 mg/day, 65-70 mg/day, 70-75 mg/day, 75-80 mg/day, 80-85 mg/day, 85-90 mg/day, 90-95 mg/day or 95-100 mg/day.
51. The method of any one of claims 43 to 50, wherein the mTOR inhibitor and/or 2-Bromo-LSD are administered intravenously, intramuscularly, intraperitoneally, orally, via inhalation or transdermally.
52. A method of lessening or alleviating severity of a major depressive disorder in a subject in need thereof, wherein the subject has been diagnosed with the major depressive disorder, comprising:
   administering a therapeutically effective amount of a mTOR inhibitor to the subject; and
   administering a therapeutically effective amount of 2-Bromo-LSD to the subject,
   wherein the mTOR inhibitor is selected from the group consisting of (i) temsirolimus (CCI-779) or a pharmaceutical equivalent, analog, derivative or a salt thereof, (ii) Everolimus (RAD001) or a pharmaceutical equivalent, analog, derivative or a salt thereof, and (iii) Rapamycin or a pharmaceutical equivalent, analog, derivative or a salt thereof.
53. The method of claim 52, wherein the mTOR inhibitor and the 2-Bromo-LSD are administered concurrently.
54. The method of claim 52, wherein the mTOR inhibitor and the 2-Bromo-LSD are administered sequentially.
55. The method of claim 52, wherein the mTOR inhibitor and the 2-Bromo-LSD are administered within 12 hours of each other.
56. The method of claim 52, wherein the mTOR inhibitor and the 2-Bromo-LSD are administered within 6 hours of each other.
57. The method of claim 52, wherein the mTOR inhibitor and the 2-Bromo-LSD are administered within 4 hours of each other.
58. The method of claim 52, wherein the mTOR inhibitor and the 2-Bromo-LSD are administered within 2 hours of each other.
59. The method of any one of claims 52 to 58, wherein the effective amount of the mTOR inhibitor is 0.1-0.5 mg/day. 0.5-1.0 mg/day, 1.0-1.5 mg/day, 1.5-2 mg/day, 2.0-2.5 mg/day, 2.5-5 mg/day, 5-10 mg/day, 10-15 mg/day, 15-20 mg/day, 20-25 mg/day, 25-30 mg/day, 30-35 mg/day, 35-40 mg/day, 40-45 mg/day, 45-50 mg/day, 50-55 mg/day, 55-60 mg/day, 60-65 mg/day, 65-70 mg/day, 70-75 mg/day, 75-80 mg/day, 80-85 mg/day, 85-90 mg/day, 90-95 mg/day or 95-100 mg/day.
60. The method of any one of claims 52 to 59, wherein the mTOR inhibitor and/or 2-Bromo-LSD are administered intravenously, intramuscularly, intraperitoneally, orally, via inhalation or transdermally.
61. A method of treating a mental disorder or a mood disorder comprising co-administering to a subject in need of treatment thereof (i) a therapeutically effective amount of a pharmaceutical composition comprising 2-Bromo-LSD; and (ii) a therapeutically effective amount of a pharmaceutical composition comprising a mTOR inhibitor.
   wherein the mental disorder and/or the mood disorder is selected from the group consisting of Attention Deficit Hyperactivity Disorder, Obsessive-Compulsive Disorder, Atypical Depression, Melancholid Depression, Psychotic major depression, Catatonic Depression, Postpartum Depression, Premenstrual dysphoric disorder, Seasonal affective disorder, Dysthymia Double Depression, Depressive Disorder Not Otherwise Specified, Depressive personality disorder, Recurrent brief depression, and Minor Depressive disorder.
62. The method of claim 61, wherein the therapeutically effective amount of the 2-Bromo-LSD is administered at least once per day.
63. The method of claim 61, wherein the therapeutically effective amount of the 2-Bromo-LSD is about 25 μg to about 50 μg.
64. The method of claim 61, wherein the therapeutically effective amount of the 2-Bromo-LSD is about 30 μg to about 45 μg.
65. The method of any one of claims 61 to 64, wherein the effective amount of the mTOR inhibitor is 0.1-0.5 mg/day. 0.5-1.0 mg/day, 1.0-1.5 mg/day, 1.5-2 mg/day, 2.0-2.5 mg/day, 2.5-5 mg/day, 5-10 mg/day, 10-15 mg/day, 15-20 mg/day, 20-25 mg/day, 25-30 mg/day, 30-35 mg/day, 35-40 mg/day, 40-45 mg/day, 45-50 mg/day, 50-55 mg/day, 55-60 mg/day, 60-65 mg/day, 65-70 mg/day, 70-75 mg/day, 75-80 mg/day, 80-85 mg/day, 85-90 mg/day, 90-95 mg/day or 95-100 mg/day.
66. The method of any one of claims 61 to 64, wherein the mTOR inhibitor and/or 2-Bromo-LSD are administered intravenously, intramuscularly, intraperitoneally, orally, via inhalation or transdermally.
67. The method of any one of claims 61 to 66, wherein the mTOR inhibitor is Rapamycin.
68. The method of any one of claims 61 to 66, wherein the mTOR inhibitor is a prodrug of Rapamycin.
69. The method of any one of claims 61 to 66, wherein the mTOR inhibitor is temsirolimus.
70. The method of any one of claims 61 to 66, wherein the mTOR inhibitor is Everolimus.
71. A method of prolonging an antidepressant effect of 2-Bromo-LSD, comprising: administering a therapeutically effective amount of a mTOR inhibitor to a subject; and administering a therapeutically effective amount of 2-Bromo-LSD to the subject, wherein the antidepressant effect of the 2-Bromo-LSD is extended
72. A composition comprising: (i) a therapeutically effective dose of an mTOR inhibitor; (ii) a therapeutically effective dose of 2-Bromo-LSD; and (iii) a pharmaceutically acceptable carrier or excipient.
73. The composition of claim 71, formulated for intravenous, intramuscular, intraperitoneal, oral, inhalation or transdermal administration.
74. A kit comprising: (i) a therapeutically effective dose of an mTOR inhibitor; and (ii) a therapeutically effective dose of 2-Bromo-LSD, and optional instructions for use.
75. The kit of claim 74, wherein the mTOR inhibitor is separately formulated from said 2-bromo-LSD.
76. The kit of claim 74, wherein the mTOR inhibitor is formulated together with said 2-bromo-LSD as a combination dosage form.
77. The kit of claim 76, comprising a plurality of said combination dosage form.
78. A capsule comprising about 2.0 mg to about 3.5 mg 2-bromo-LSD, about 296.5 to about 298 mg microcrystalline cellulose, and about 3 mg silicon dioxide or magnesium stearate.
79. A tablet comprising about 2.0 mg to about 3.5 mg 2-bromo-LSD, about 296.5 to about 298 mg microcrystalline cellulose, and about 3 mg silicon dioxide or magnesium stearate.
80. A oral dissolving tablet comprising about 2.0 mg to about 3.5 mg 2-bromo-LSD, about 1.0 mg to about 162.5 mg microcrystalline cellulose, about 1.0 mg to about 162.5 mg D-mannitol, about 1.0 mg to about 162.5 mg xylitol, about 1.0 mg to about 162.5 mg crospovidone, about 1.0 mg to about 162.5 mg dibasic calcium phosphate anhydrous and about 1.25 mg magnesium stearate,
   wherein said oral dissolving tablet has a compression range of about 34 to about 40 Newtons (N), and
   wherein said oral dissolving tablet has a disintegration time of fewer than about 30 seconds.
81. The capsule of claim 78, the tablet of claim 79 or the oral dissolving tablet of claim 80 further comprising a mTOR inhibitor.
82. The capsule of claim 78, the tablet of claim 79 or the oral dissolving tablet of claim 80 for use with a mTOR inhibitor, wherein the mTOR inhibitor is formulated as a transdermal patch for continuous administration.
83. A composition for use in the treatment of a mental or mood disorder, the composition comprising:
   a therapeutically effective dose of an mTOR inhibitor, and
   a pharmaceutically acceptable carrier or excipient.
84. The composition for use of claim 83, further comprising use of an ointment comprising about 0.03% to about 0.1% (w/w) mTOR inhibitor in an ointment base.
85. The composition for use of claim 84, wherein the mTOR inhibitor is selected from the group consisting of temsirolimus (CCI-779), Everolimus (RAD001), Rapamycin and pharmaceutical equivalents, analogs, derivatives or salts thereof.

The present invention is further illustrated by the following examples, which should not be construed as limiting in any way. In the below examples, a mTOR inhibitor can be further incorporated. One skilled in the art will appreciate that the constituents of the formulation may be varied in amounts yet continue to be within the spirit and scope of the present invention. One skilled in the art will appreciate that the constituents of the formulations may be varied in amounts yet continue to be within the spirit and scope of the present invention.

The following examples are provided for exemplification of the present invention and are not intended to be limiting in any way.

### EXAMPLES

### Example 1—Oral Dosing in a Capsule

| Oral Dosing in a Capsule | mg |
|---|---|
| 2-Bromo-LSD (based on patient body weight) | about 2.0 to about 3.5 |
| Microcrystalline Cellulose | about 296.5 to about |
| | 298.0 |
| Lubricant, Silicon Dioxide or Magnesium Stearate | about 3 |

### Example 2—Oral Dosing in a Tablet

Tablets have a range of hardness at 100+/−50 Newtons

| Oral Dosing in a Tablet | mg |
|---|---|
| 2-Bromo-LSD (based on patient body weight) | about 2.0 to about 3.5 |
| Microcrystalline Cellulose | about 296.5 to about |
| | 298.0 |
| Lubricant, Silicon Dioxide or Magnesium Stearate | about 3 |

### Example 3—Oral Dissolving Table

Tablets were pressed on a rotary tablet press to a compression range of about 34 to about 40 Newtons (N). At this tablet hardness, all tablets had a disintegration time of fewer than about 30 seconds.

| Oral Dissolving Tablet | mg | |
|---|---|---|
| D-Mannitol | about 1.0 to about 162.5 | Diluent & |
| | | Palatability |
| Xylitol | about 1.0 to about 162.5 | Sweetener |
| Microcrystalline Cellulose | about 1.0 to about 162.5 | Binder |
| Crospovidone | about 1.0 to about 162.5 | Disintegrant |
| Dibasic Calcium | about 1.0 to | Functional |
| Phosphate Anhydrous | about 162.5 | Filler |
| 2-Bromo-LSD (based on | about 2.0 to | Active |
| patient body weight) | about 3.5 | |
| Magnesium Stearate | about 1.25 | Lubricant |
| 0.5% by weight | | |

### Example 4—Oral Dosing in Solution

| Oral Dosing in solution | mg | ml |
|---|---|---|
| 2-Bromo-LSD (based on | about 2.0 to | |
| patient body weight) | about 3.5 | |
| Distilled Water | | about 120 |

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. A method of treating major depressive disorder comprising administering to a subject a therapeutically effective amount of 2-Bromo-LSD or a derivative or salt thereof.

2. The method of claim 1, wherein the therapeutically effective amount of the 2-Bromo-LSD is administered at least once per day.

3. The method of claim 1 or 2, wherein the therapeutically effective amount is up to 50 μg or about 50 μg of said 2-Bromo-LSD or the derivative or salt thereof.

4. The method of claim 3, wherein the therapeutically effective amount is about 27 μg to about 33 μg of said 2-Bromo-LSD or the derivative or salt thereof.

5. The method of claim 4, wherein the therapeutically effective amount is about 30 μg of said 2-Bromo-LSD or the derivative or salt thereof.

6. The method of any one of claims 1 to 5, wherein the 2-Bromo-LSD or the derivative or salt thereof is formulated as a pharmaceutical composition further comprising a pharmaceutically acceptable carrier or excipient.

7. The method of claim 6, wherein the pharmaceutical composition is formulated for intravenous, intramuscular, intraperitoneal, oral, or inhalation administration.

8. The method of claim 7, wherein the pharmaceutical composition is formulated as a tablet or as a capsule.

9. The method of claim 6 or 7, wherein the pharmaceutical composition is formulated as an oral dosing solution.

10. The method of any one of claims 1 to 9, further comprising the administration of a mTOR inhibitor prior to or with said 2-Bromo-LSD or a derivative or salt thereof, optionally wherein said mTOR inhibitor is formulated in a same or a different formulation format to said 2-Bromo-LSD.

11. A method of treating a mental disorder and/or a mood disorder comprising administering to a subject a therapeutically effective amount of 2-Bromo-LSD or a derivative or salt thereof,
wherein the mental disorder and/or the mood disorder is selected from the group consisting of Attention Deficit Hyperactivity Disorder, Obsessive-Compulsive Disorder, Atypical Depression, Melancholid Depression, Psychotic major depression, Catatonic Depression, Postpartum Depression, Premenstrual dysphoric disorder, Seasonal affective disorder, Dysthymia Double Depression, Depressive Disorder Not Otherwise Specified, Depressive personality disorder, Recurrent brief depression, and Minor Depressive disorder.

12. The method of claim 11, wherein the therapeutically effective amount of the 2-Bromo-LSD is administered at least once per day.

13. The method of claim 11 or 12, wherein the therapeutically effective amount is up to about 35 μg of said 2-Bromo-LSD or the derivative or salt thereof.

14. The method of claim 13, wherein the therapeutically effective amount is about 15 μg to about 30 μg of said 2-Bromo-LSD or the derivative or salt thereof.

15. The method of claim 14, wherein the therapeutically effective amount is about 30 μg of said 2-Bromo-LSD or the derivative or salt thereof.

16. The method of any one of claims 11 to 15, wherein the 2-Bromo-LSD or a derivative or salt thereof is formulated as a pharmaceutical composition further comprising a pharmaceutically acceptable carrier or excipient.

17. The method of claim 16, wherein the pharmaceutical composition is formulated for intravenous, intramuscular, intraperitoneal, oral, or inhalation administration.

18. The method of claim 17, wherein the pharmaceutical composition is formulated as a tablet or as a capsule.

19. The method of claim 16 or 17, wherein the pharmaceutical composition is formulated as an oral dosing solution.

20. The method of any one of claims 11 to 19, further comprising the administration of a mTOR inhibitor prior to, concurrently with, or within set times with said 2-Bromo-LSD or the derivative or salt thereof, optionally wherein said mTOR inhibitor is formulated as the same or a different formulation format to said 2-Bromo-LSD.

21. A method of treating a bipolar disorder comprising administering to a subject a therapeutically effective amount of 2-Bromo-LSD or a derivative or salt thereof.

22. The method of claim 21, wherein the therapeutically effective amount of the 2-Bromo-LSD is administered at least once per day.

23. The method of claim 21 or 22, wherein the therapeutically effective amount is up to about 35 μg of said 2-Bromo-LSD or the derivative or salt thereof.

24. The method of claim 23, wherein the therapeutically effective amount is about 27 μg to about 33 μg of said 2-Bromo-LSD or the derivative or salt thereof.

25. The method of claim 24, wherein the therapeutically effective amount is about 30 μg of said 2-Bromo-LSD or the derivative or salt thereof.

26. The method of any one of claims 21 to 25, wherein the 2-Bromo-LSD or the derivative or salt thereof is formulated as a pharmaceutical composition further comprising a pharmaceutically acceptable carrier or excipient.

27. The method of claim 26, wherein the pharmaceutical composition is formulated for intravenous, intramuscular, intraperitoneal, oral, or inhalation administration.

28. The method of claim 27, wherein the pharmaceutical composition is formulated as a tablet or as a capsule.

29. The method of claim 16 or 17, wherein the pharmaceutical composition is formulated as an oral dosing solution.

30. The method of any one of claims 21 to 29, further comprising the administration of a mTOR inhibitor prior to, concurrently with, or within set times with said 2-Bromo-LSD or the derivative or salt thereof.

31. The method of claim 30, wherein said mTOR inhibitor is formulated as the same or in a different formulation format to said 2-Bromo-LSD.

32. The method of any one of claims 21 to 31, wherein the bipolar disorder is selected from the group consisting of bipolar I, bipolar II, cyclothymia, and bipolar disorder not otherwise specified.

33. A method of treating a mental disorder and/or a mood disorder comprising administering to a subject a therapeutically effective amount of 2-Bromo-LSD or a derivative or salt thereof, wherein the mental disorder and/or the mood disorder is selected from the group consisting of impulse-control disorders, adjustment disorders, and personality disorders.

34. The method of claim 33, wherein the therapeutically effective amount of the 2-Bromo-LSD is administered at least once per day.

35. The method of claim 33 or 34, wherein the therapeutically effective amount is up to about 50 μg of said 2-Bromo-LSD or the derivative or salt thereof.

36. The method of claim 35, wherein the therapeutically effective amount is about 25 μg to about 50 μg of said 2-Bromo-LSD or the derivative or salt thereof.

37. The method of claim 36, wherein the therapeutically effective amount is about 30 μg to about 45 μg of said 2-Bromo-LSD or the derivative or salt thereof.

38. The method of any one of claims 33 to 37, wherein the 2-Bromo-LSD or a derivative or salt thereof is formulated as a pharmaceutical composition further comprising a pharmaceutically acceptable carrier or excipient.

39. The method of claim 38, wherein the pharmaceutical composition is formulated for intravenous, intramuscular, intraperitoneal, oral, or inhalation administration.

40. The method of claim 39, wherein the pharmaceutical composition is formulated as a tablet or as a capsule.

41. The method of claim 38 or 39, wherein the pharmaceutical composition is formulated as an oral dosing solution.

42. The method of any one of claims 33 to 41, further comprising the administration of a mTOR inhibitor prior to, concurrently with, or within set times with said 2-Bromo-LSD or the derivative or salt thereof, optionally wherein said mTOR inhibitor is formulated as the same or a different formulation format to said 2-Bromo-LSD.

43. A method of treating major depressive disorder in a subject in need thereof, wherein the subject has been diagnosed with the major depressive disorder, comprising:
administering a therapeutically effective amount of a mTOR inhibitor and a therapeutically effective amount of 2-Bromo-LSD to the subject,
wherein the mTOR inhibitor is selected from the group consisting of (i) temsirolimus (CCI-779) or a pharmaceutical equivalent, analog, derivative or a salt thereof, (ii) Everolimus (RAD001) or a pharmaceutical equivalent, analog, derivative or a salt thereof, and (iii) Rapamycin or a pharmaceutical equivalent, analog, derivative or a salt thereof.

44. The method of claim 43, wherein the mTOR inhibitor and the 2-Bromo-LSD are administered concurrently.

45. The method of claim 43, wherein the mTOR inhibitor and the 2-Bromo-LSD are administered sequentially.

46. The method of claim 43, wherein the mTOR inhibitor and the 2-Bromo-LSD are administered within 12 hours of each other.

47. The method of claim 43, wherein the mTOR inhibitor and the 2-Bromo-LSD are administered within 6 hours of each other.

48. The method of claim 43, wherein the mTOR inhibitor and the 2-Bromo-LSD are administered within 4 hours of each other.

49. The method of claim 43, wherein the mTOR inhibitor and the 2-Bromo-LSD are administered within 2 hours of each other.

50. The method of any one of claims 43 to 49, wherein the effective amount of the mTOR inhibitor is 0.1-0.5 mg/day, 0.5-1.0 mg/day, 1.0-1.5 mg/day, 1.5-2 mg/day, 2.0-2.5 mg/day, 2.5-5 mg/day, 5-10 mg/day, 10-15 mg/day, 15-20 mg/day, 20-25 mg/day, 25-30 mg/day, 30-35 mg/day, 35-40 mg/day, 40-45 mg/day, 45-50 mg/day, 50-55 mg/day, 55-60 mg/day, 60-65 mg/day, 65-70 mg/day, 70-75 mg/day, 75-80 mg/day, 80-85 mg/day, 85-90 mg/day, 90-95 mg/day or 95-100 mg/day.

51. The method of any one of claims 43 to 50, wherein the mTOR inhibitor and/or 2-Bromo-LSD are administered intravenously, intramuscularly, intraperitoneally, orally, via inhalation or transdermally.

52. A method of lessening or alleviating severity of a major depressive disorder in a subject in need thereof, wherein the subject has been diagnosed with the major depressive disorder, comprising:
administering a therapeutically effective amount of a mTOR inhibitor to the subject; and
administering a therapeutically effective amount of 2-Bromo-LSD to the subject,
wherein the mTOR inhibitor is selected from the group consisting of (i) temsirolimus (CCI-779) or a pharmaceutical equivalent, analog, derivative or a salt thereof, (ii) Everolimus (RAD001) or a pharmaceutical equivalent, analog, derivative or a salt thereof, and (iii) Rapamycin or a pharmaceutical equivalent, analog, derivative or a salt thereof.

53. The method of claim 52, wherein the mTOR inhibitor and the 2-Bromo-LSD are administered concurrently.

54. The method of claim 52, wherein the mTOR inhibitor and the 2-Bromo-LSD are administered sequentially.

55. The method of claim 52, wherein the mTOR inhibitor and the 2-Bromo-LSD are administered within 12 hours of each other.

56. The method of claim 52, wherein the mTOR inhibitor and the 2-Bromo-LSD are administered within 6 hours of each other.

57. The method of claim 52, wherein the mTOR inhibitor and the 2-Bromo-LSD are administered within 4 hours of each other.

58. The method of claim 52, wherein the mTOR inhibitor and the 2-Bromo-LSD are administered within 2 hours of each other.

59. The method of any one of claims 52 to 58, wherein the effective amount of the mTOR inhibitor is 0.1-0.5 mg/day, 0.5-1.0 mg/day, 1.0-1.5 mg/day, 1.5-2 mg/day, 2.0-2.5 mg/day, 2.5-5 mg/day, 5-10 mg/day, 10-15 mg/day, 15-20 mg/day, 20-25 mg/day, 25-30 mg/day, 30-35 mg/day, 35-40 mg/day, 40-45 mg/day, 45-50 mg/day, 50-55 mg/day, 55-60 mg/day, 60-65 mg/day, 65-70 mg/day, 70-75 mg/day, 75-80 mg/day, 80-85 mg/day, 85-90 mg/day, 90-95 mg/day or 95-100 mg/day.

60. The method of any one of claims 52 to 59, wherein the mTOR inhibitor and/or 2-Bromo-LSD are administered intravenously, intramuscularly, intraperitoneally, orally, via inhalation or transdermally.

61. A method of treating a mental disorder or a mood disorder comprising co-administering to a subject in need of treatment thereof (i) a therapeutically effective amount of a pharmaceutical composition comprising 2-Bromo-LSD; and (ii) a therapeutically effective amount of a pharmaceutical composition comprising a mTOR inhibitor.
wherein the mental disorder and/or the mood disorder is selected from the group consisting of Attention Deficit Hyperactivity Disorder, Obsessive-Compulsive Disorder, Atypical Depression, Melancholid Depression, Psychotic major depression, Catatonic Depression, Postpartum Depression, Premenstrual dysphoric disorder, Seasonal affective disorder, Dysthymia Double Depression, Depressive Disorder Not Otherwise Specified, Depressive personality disorder, Recurrent brief depression, and Minor Depressive disorder.

62. The method of claim 61, wherein the therapeutically effective amount of the 2-Bromo-LSD is administered at least once per day.

63. The method of claim 61, wherein the therapeutically effective amount of the 2-Bromo-LSD is about 25 μg to about 50 μg.

64. The method of claim 61, wherein the therapeutically effective amount of the 2-Bromo-LSD is about 30 μg to about 45 μg.

65. The method of any one of claims 61 to 64, wherein the effective amount of the mTOR inhibitor is 0.1-0.5 mg/day, 0.5-1.0 mg/day, 1.0-1.5 mg/day, 1.5-2 mg/day, 2.0-2.5 mg/day, 2.5-5 mg/day, 5-10 mg/day, 10-15 mg/day, 15-20 mg/day, 20-25 mg/day, 25-30 mg/day, 30-35 mg/day, 35-40 mg/day, 40-45 mg/day, 45-50 mg/day, 50-55 mg/day, 55-60 mg/day, 60-65 mg/day, 65-70 mg/day, 70-75 mg/day, 75-80 mg/day, 80-85 mg/day, 85-90 mg/day, 90-95 mg/day or 95-100 mg/day.

66. The method of any one of claims 61 to 64, wherein the mTOR inhibitor and/or 2-Bromo-LSD are administered intravenously, intramuscularly, intraperitoneally, orally, via inhalation or transdermally.

67. The method of any one of claims 61 to 66, wherein the mTOR inhibitor is Rapamycin.

68. The method of any one of claims 61 to 66, wherein the mTOR inhibitor is a prodrug of Rapamycin.

69. The method of any one of claims 61 to 66, wherein the mTOR inhibitor is temsirolimus.

70. The method of any one of claims 61 to 66, wherein the mTOR inhibitor is Everolimus.

71. A method of prolonging an antidepressant effect of 2-Bromo-LSD, comprising:
administering a therapeutically effective amount of a mTOR inhibitor to a subject; and
administering a therapeutically effective amount of 2-Bromo-LSD to the subject,
wherein the antidepressant effect of the 2-Bromo-LSD is extended

72. A composition comprising: (i) a therapeutically effective dose of an mTOR inhibitor; (ii) a therapeutically effective dose of 2-Bromo-LSD; and (iii) a pharmaceutically acceptable carrier or excipient.

73. The composition of claim 71, formulated for intravenous, intramuscular, intraperitoneal, oral, inhalation or transdermal administration.

74. A kit comprising: (i) a therapeutically effective dose of an mTOR inhibitor; and (ii) a therapeutically effective dose of 2-Bromo-LSD, and optional instructions for use.

75. The kit of claim 74, wherein the mTOR inhibitor is separately formulated from said 2-bromo-LSD.

76. The kit of claim 74, wherein the mTOR inhibitor is formulated together with said 2-bromo-LSD as a combination dosage form.

77. The kit of claim 76, comprising a plurality of said combination dosage form.

78. A capsule comprising about 2.0 mg to about 3.5 mg 2-bromo-LSD, about 296.5 to about 298 mg microcrystalline cellulose, and about 3 mg silicon dioxide or magnesium stearate.

79. A tablet comprising about 2.0 mg to about 3.5 mg 2-bromo-LSD, about 296.5 to about 298 mg microcrystalline cellulose, and about 3 mg silicon dioxide or magnesium stearate.

80. A oral dissolving tablet comprising about 2.0 mg to about 3.5 mg 2-bromo-LSD, about 1.0 mg to about 162.5 mg microcrystalline cellulose, about 1.0 mg to about 162.5 mg D-mannitol, about 1.0 mg to about 162.5 mg xylitol, about 1.0 mg to about 162.5 mg crospovidone, about 1.0 mg to about 162.5 mg dibasic calcium phosphate anhydrous and about 1.25 mg magnesium stearate,
wherein said oral dissolving tablet has a compression range of about 34 to about 40 Newtons (N), and
wherein said oral dissolving tablet has a disintegration time of fewer than about 30 seconds.

81. The capsule of claim 78, the tablet of claim 79 or the oral dissolving tablet of claim 80 further comprising a mTOR inhibitor.

82. The capsule of claim 78, the tablet of claim 79 or the oral dissolving tablet of claim 80 for use with a mTOR inhibitor, wherein the mTOR inhibitor is formulated as a transdermal patch for continuous administration.

83. A composition for use in the treatment of a mental or mood disorder, the composition comprising:
a therapeutically effective dose of an mTOR inhibitor, and
a pharmaceutically acceptable carrier or excipient.

84. The composition for use of claim 83, further comprising use of an ointment comprising about 0.03% to about 0.1% (w/w) mTOR inhibitor in an ointment base.

85. The composition for use of claim 84, wherein the mTOR inhibitor is selected from the group consisting of temsirolimus (CCI-779), Everolimus (RAD001), Rapamycin and pharmaceutical equivalents, analogs, derivatives or salts thereof.
